(19) United States
(12) Patent Application Publication
(10) Pub. No.: US 2010/0056563 A1
(43) Pub. Date: Mar. 4, 2010

(54) NOVEL 1.8-NAPHTHYRIDINE COMPOUNDS

(76) Inventors: Deodialsingh Guiadeen, Chesterfield, NJ (US); Jeffrey J. Hale, Westfield, NJ (US); Shankaran Kothandaraman, Kendall Park, NJ (US)

Correspondence Address: MERCK AND CO., INC, P O BOX 2000, RAHWAY, NJ, 07065-0907, US

(21) Appl. No.: 12/594,849

(22) PCT Filed: Apr. 14, 2008
(86) PCT No.: PCT/US08/04817

§ 371 Date: Oct. 6, 2009

Related U.S. Application Data
(60) Provisional application No. 60/925,019, filed on Apr. 18, 2007.

(51) Int. Cl. A61K 31/4375; C07D 471/04; A61P 43/00

(52) U.S. Cl. 514/300; 546/123

(57) ABSTRACT

The present invention relates to naphthyridine compounds useful as HIF prolyl hydroxylase inhibitors to treat anemia and like conditions.

### BACKGROUND OF THE INVENTION

The insufficient delivery of oxygen to cells and tissues is associated with anemia, which is defined as a deficiency in the blood's oxygen-carrying capacity, and ischemia, in which restrictions in blood supply are caused by a constriction or blockage of blood vessels. Anemia can be caused by the loss of red blood cells (hemorrhage), excessive red blood cell destruction (hemolysis) or deficiencies in erythropoiesis (production of red blood cells from precursors found in the bone marrow). The symptoms of anemia can include weakness, dizziness, fatigue, pallor, impairment of cognitive function and a general reduction in quality of life. Chronic and/or severe anemia can lead to the exacerbation of myocardial, cerebral or peripheral ischemia and to heart failure. Ischemia is defined as an absolute or relative shortage of oxygen to a tissue or organ and can result from disorders such as atherosclerosis, diabetes, thromboembolisms, hypotension, etc. The heart, brain and kidney are especially sensitive to ischemic stress caused by low blood supply.

The primary pharmacological treatment for anemia is administration of some variant of recombinant human erythropoietin (EPO). For anemias associated with kidney disease, chemotherapy-induced anemia, anemia from HIV-therapy or anemia due to blood loss, recombinant EPO is administered to enhance the supply of the hormone, correct the shortage of red blood cells and increase the blood's oxygen-carrying capacity. EPO replacement is not always sufficient to stimulate optimal erythropoiesis (e.g., in patients with iron processing deficiencies) and has associated risks.

Hypoxia-inducible factor (HIF) has been identified as a primary regulator of the cellular response to low oxygen. HIF is a heterodimeric gene transcription factor consisting of a highly regulated α-subunit (HIF-α) and a constitutively expressed β-subunit (HIF-β, also known as ARNT, or aryl hydrocarbon receptor nuclear transporter). HIF target genes are reported to be associated with various aspects of erythropoiesis (e.g., erythropoietin (EPO) and EPO receptor), glycolysis and angiogenesis (e.g., vascular endothelial growth factor (VEGF)). Genes for proteins involved in iron absorption, transport and utilization as well as heme synthesis are also targets of HIF.

Under normal oxygenation, HIF-α is a substrate in a reaction with molecular oxygen, which is catalyzed by a family of iron(II)-, 2-ketoglutarate- and ascorbate-dependent dioxygenase enzymes called PHD-1 (EGLN2, or egg laying abnormal 9 homolog 2, PHD2 (EGLN1), and PHD3 (EGLN3). Proline residues of HIF-α are hydroxylated (e.g., Pro-402 and Pro-564 of HIF-1α) and the resulting product is a target of the tumor suppressor protein von-Hippel Lindau, a component of an E3 ubiquitin ligase multiprotein complex involved in protein ubiquitination. Under low oxygenation, the HIF-α hydroxylation reaction is less efficient and HIF-α is available to dimerize with HIF-\tilde{β} HIF dimers are translocated to the cell nucleus where they bind to a hypoxia-responsive enhancer element of HIF target genes.

Cellular levels of HIF are known to increase under conditions of hypoxia and after exposure to hypoxia mimetic agents. The latter includes, but is not limited to, specific metal ions (e.g., cobalt, nickel, manganese), iron chelators (e.g., desferrioxamine) and analogs of 2-ketoglurate (e.g., N-oxalyl glycine). The compounds of the present invention inhibit the HIF prolyl hydroxylases (PHD-1, PHD-2, PHD-3) and can also serve to modulate HIF levels. These compounds therefore have utility for the treatment and/or prevention of disorders or conditions where HIF modulation is desirable, such as anemia and ischemia. As an alternative to recombinant erythropoietin therapy, the compounds of the present invention provide a simpler and broader method for the management of anemia.

### SUMMARY OF THE INVENTION

The present invention concerns compounds which inhibit HIF prolyl hydroxylase, their use for enhancing endogenous production of erythropoietin, and for treating conditions associated with reduced endogenous production of erythropoietin such as anemia and like conditions, as well as pharmaceutical compositions comprising such a compound and a pharmaceutical carrier.

### DETAILED DESCRIPTION OF THE INVENTION

In one aspect the present invention provides compounds having the formula I:

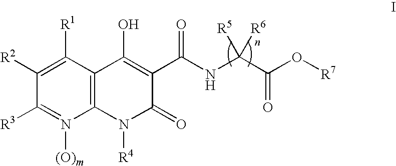

or a pharmaceutically acceptable salt or a solvate thereof, wherein
m is 0 or 1;
n is 1 or 2;
p is 0, 1 or 2;
R¹, R² and R³ are independently selected from the group consisting of:
i) hydrogen,
ii) —C₁-C₁₀ alkyl, optionally substituted with one to five groups independently selected from R^{a}, iii) —C₃-C₁₀ cycloalkyl, optionally substituted with one to five groups independently selected from R^{a},
iv) —C₂-C₁₀ alkenyl, optionally substituted with one to five groups independently selected from R^{a},
v) —C₅-C₁₀ cycloalkenyl, optionally substituted with one to five groups independently selected from R^{a},
vi) —C₂-C₁₀ alkynyl, optionally substituted with one to five groups independently selected from R^{a},
vii) aryl, optionally substituted with one to three groups independently selected from R^{b} and hydroxy,
viii) halogen,
ix) cyano,
x) heteroaryl, optionally substituted with one to three groups independently selected from R^{b},
xi) —O—C₁-C₁₀ alkyl, optionally substituted with one to five groups independently selected from fluorine, hydroxy, oxo, cyano, aryl, substituted aryl, heteroaryl, substituted heteroaryl, —C₁-C₆ alkoxy, aryloxy, substituted aryloxy, heteroaryloxy, substituted heteroaryloxy, —CO₂R⁷, —NR⁸R⁹, —CONR⁸R⁹, —OCO₂R⁷, —OCONR⁸R⁹, —NR¹⁰CO₂R⁷, —NR¹⁰CONR⁸R⁹, and —S(O)ₚR¹⁰;
xii) —O-aryl, optionally substituted with one to three groups independently selected from R^{b} and hydroxy,
xiii) —O-heteroaryl, optionally substituted with one to three groups independently selected from R^{b};
xiv) —SOₚ—C₁-C₁₀ alkyl, optionally substituted with one to five groups independently selected from R^{a};
xvi) —SOₚ-aryl, optionally substituted with one to three groups independently selected from hydroxy and R^{b}; or
R¹ and R², or R² and R³ are joined to form a ring of 5 to 8 atoms optionally substituted with one to three groups independently selected from fluorine, phenyl, substituted phenyl, heteroaryl, substituted heteroaryl, —CONR⁸R⁹, —CO₂R³, and —NR⁸R⁹; where said ring is partially or fully unsaturated having 0, 1 or 2 heteroatoms independently selected from —NR⁷—, —O— and —S(O)ₚ—;
R⁴ is selected from the group consisting of:
i) hydrogen;
ii) —C₁-C₁₀ alkyl, optionally substituted with one to five groups independently selected from R^{a};
iii) —(C₀-C₁₀ alkyl)C₃-C₁₀ cycloalkyl, optionally substituted with one to five groups independently selected from R^{a},
iv) —C₂-C₁₀ alkenyl, optionally substituted with one to five groups independently selected from R^{a};
v) —(C₀-C₁₀ alkyl)C₅-C₁₀ cycloalkenyl, optionally substituted with one to five groups independently selected from R^{a};
vi) —C₂-C₁₀ alkynyl optionally substituted with one to five groups independently selected from R^{a};
vii) —(C₀-C₁₀ alkyl)aryl, optionally substituted with one to three groups independently selected from hydroxy and R^{b}; and
ix) —(C₀-C₁₀ alkyl)heteroaryl, optionally substituted with one to three groups independently selected from R^{b};
R⁵ and R⁶ are independently selected from the group consisting of:
i) hydrogen;
ii) C₁-C₄ alkyl, optionally substituted with a hydroxy, —SH, —NH2 or CO₂H;
iii) trifluoromethyl; and
iv) 2,2,2-trifluoroethyl;
R⁷ is selected from the group consisting of:
i) hydrogen;
ii) —C₁-C₁₀ alkyl;
iii) —(CH₂)₁₋₆—C₃-C₈ cycloalkyl; and
iv) —(CH₂)₁₋₆phenyl;
R⁸, R⁹ and R¹⁰ are independently selected from the group consisting of:
i) hydrogen;
ii) —C₁-C₆ alkyl;
iii) —C₃-C₆ cycloalkyl, wherein alkyl and cycloalkyl are each optionally substituted with one to five groups independently selected from fluorine, hydroxy, oxo, cyano, aryl, substituted aryl, heteroaryl, substituted heteroaryl, —C₁-C₆ alkoxy, substituted —C₁-C₆ alkoxy, aryloxy, substituted aryloxy, heteroaryloxy, substituted heteroaryloxy, —S(O)ₚalkyl and —S(O)ₚaryl;
iv) aryl, optionally substituted with one to three groups independently selected from C₁-C₆ alkyl, halogen, hydroxy, cyano, —CO₂(C₁₋₃alkyl), —CONR¹¹R¹², —OCO₂(C₁₋₃alkyl), —OCONR¹¹R¹², and —S(O)ₚ(C₁₋₃alkyl); and
v) heteroaryl, optionally substituted with one three groups independently selected from C₁-C₆ alkyl, halogen, hydroxy, oxo, cyano, —CO₂(C₁₋₃alkyl), —CONR¹¹R¹², —OCO₂(C₁₋₃alkyl), —OCONR¹¹R¹², and —S(O)ₚ(C₁₋₃alkyl); or
R⁸ and R⁹ together with the N atom to which they are attached form a saturated or partially saturated ring of 5 to 8 atoms having 0, 1 or 2 additional heteroatoms selected from —O—, —NR⁷—, and —S(O)ₚ— wherein said ring is optionally substituted with a methyl or hydroxy group;
R¹¹ and R¹² are independently selected from the group consisting of:
i) hydrogen;
ii) C₁-C₄ alkyl, optionally substituted with a hydroxy; or
R¹¹ and R¹² together with the N atom to which they are attached form a saturated or partially saturated ring of 5 to 8 atoms having 0, 1 or 2 additional heteroatoms selected from —O—, —NR⁷—, and —S(O)ₚ—;
R^{a} is selected from the group consisting of fluorine, hydroxy, oxo, cyano, aryl, substituted aryl, heteroaryl, substituted heteroaryl, —C₁-C₆ alkoxy, substituted —C₁-C₆ alkoxy, aryloxy, substituted aryloxy, heteroaryloxy, substituted heteroaryloxy, —CO₂R⁷, —NR⁸R⁹, —CONR⁸R⁹, —OCO₂R⁷, —OCONR⁸R⁹, —NR¹⁰CO₂R⁷, —NR¹⁰CONR⁸R⁹, and —S(O)ₚR¹⁰;
R^{b} is selected from the group consisting of halogen, cyano, aryl, substituted aryl, heteroaryl, substituted heteroaryl, —C₁-C₆ alkyl, substituted —C₁-C₆ alkyl, —C₁-C₆ alkoxy, substituted —C₁-C₆ alkoxy, aryloxy, substituted aryloxy, heteroaryloxy, substituted heteroaryloxy, —CO₂R⁷, —NR⁸R⁹, —CONR⁸R⁹, —OCO₂R⁷, —OCONR⁸R⁹, —NR¹⁰CO₂R⁷, —NR¹⁰CONR⁸R⁹, and —S(O)ₚR¹⁰.

In one embodiment of the invention, R¹, R² and R³ are each independently chosen from hydrogen and a halogen.

One group of compounds within Formula I are those wherein one of R¹, R² and R³ is hydrogen, and the others are independently selected from i) hydrogen, ii) C₁-C₆ alkyl optionally substituted with one to three groups independently selected from R^{a}, iii) C₃-C₈ cycloalkyl optionally substituted with one to three groups independently selected from C₁-C₄ alkyl, CF₃, and R^{a}, iv) aryl optionally substituted with one or two groups independently selected from hydroxy and R^{b}, v) halogen, vi) cyano, vii) heteroaryl optionally substituted with one or two groups independently selected from R^{b}, viii) —O—C₁-C₆ alkyl optionally substituted with one to three groups independently selected from fluorine, hydroxy, oxo, cyano, aryl, substituted aryl, heteroaryl, substituted heteroaryl, —C₁-C₆ alkoxy, aryloxy, substituted aryloxy, heteroaryloxy, substituted heteroaryloxy, —CO₂R⁷, —NR⁸R⁹, —CONR⁸R⁹, —OCO₂R⁷, —OCONR⁸R⁹, —NR¹⁰CO₂R⁷, —NR¹⁰CONR⁸R⁹, and —S(O)ₚR¹⁰; ix) —O-aryl optionally substituted with one or two groups independently selected from hydroxy and R^{b}, x) —O-heteroaryl optionally substituted with one to two groups independently selected from R^{b}; xi) —SOₚ—C₁-C₆ alkyl optionally substituted with one to three groups independently selected from R^{a}; and xii) —SOₚ-aryl optionally substituted with one to three groups independently selected from hydroxy and R^{b}. All other variables are as defined under formula I.

Within this group is a set of compounds in which R¹ is hydrogen, one of R² and R³ is hydrogen and the other is selected from the group consisting of i) hydrogen, ii) halogen, iii) cyano, iv) —C₁-C₄ alkyl optionally substituted with one to three fluorine, and iv) —O—C₁-C₄ alkyl optionally substituted with one to three fluorine. A subset of compounds are those wherein R¹ and R² are both hydrogen and R³ is selected from hydrogen, —O—C₁-C₄ alkyl, cyano and halogen. A second subset of compounds are those wherein R¹ and R³ are both hydrogen and R² is selected from hydrogen, —O—C₁-C₄ alkyl, cyano and halogen.

Another group of compounds within Formula I are those wherein R⁴ is —C₁-C₄ alkyl substituted with a group selected from C(O)OH, C(O)O—C₁-C₄ alkyl, aryl, substituted aryl, heteroaryl and substituted heteroaryl. All other variables are as defined under formula I.

Within this group is a set of compounds wherein R⁴ is —C₁-C₄ alkyl substituted with phenyl, where phenyl is unsubstituted or substituted with one to three groups independently selected from i) —C₁-C₃ alkyl optionally substituted with one to three fluorine, ii) halogen, iii) cyano, iv) C(O)NH₂, and v) —O—C₁-C₃ alkyl optionally substituted with one to three fluorine. A subset of compounds are those wherein R⁴ is benzyl wherein the phenyl moiety is optionally substituted with one or two groups independently selected from halogen, cyano, and trifluoromethyl.

A second set of compounds within this group are those wherein R⁴ is —C₁-C₄ alkyl substituted with heteroaryl, where heteroaryl is unsubstituted or substituted with one to three groups independently selected from i) —C₁-C₄ alkyl optionally substituted with one to three fluorine, ii) halogen, iii) cyano, iv) phenyl, and v) —O—C₁-C₄ alkyl optionally substituted with one to three fluorine. A subset of compounds are those where the heteroaryl is unsubstituted, monosubstituted or disubstituted and is selected from benzothiazole, benzoxazole, oxazole, isoxazole, oxadiazole (such as 1,2,4-oxadiazole and 1,3,4-oxadiazole), thiazole, isothiazole and thiadiazole (such as 1,2,4-thiadiazole and 1,3,4-thiadiazole). A further subset are compounds wherein heteroaryl and substituted heteroaryl are selected from benzothiazole, halo-substituted benzothiazole, isoxazole, phenyl substituted isoxazole, 1,2,4-oxadiazole, phenyl substituted 1,2,4-oxadiazole, thiazole, phenyl substituted thiazole, C₁-C₄ alkyl substituted thiazole, di(C₁-C₄)alkyl substituted thiazole, 1,3,4-oxadiazole, and phenyl substituted 1,3,4-oxadiazole.

Another group of compounds within Formula I are those having Formula Ia:

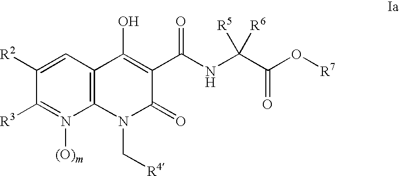

wherein m, R², R³, R⁵, R⁶ and R⁷ are as defined under Formula I, and R^{4′} is selected from C(O)OH, C(O)O—C₁-C₄ alkyl, aryl, substituted aryl, heteroaryl and substituted heteroaryl.

Within this group of Formula Ia is a set of compounds wherein R² and R³ are independently selected from hydrogen, halogen, cyano and C₁-C₃ alkyl optionally substituted with one to three fluorine. A subset of compounds are those wherein R² is hydrogen and R³ is selected from hydrogen, halogen, cyano, trifluoromethyl and trifluoromethoxy. Another subset are compounds wherein R³ is hydrogen and R² is selected from hydrogen, halogen, cyano, trifluoromethyl and trifluoromethoxy.

Within this group of Formula Ia is a second set of compounds wherein R^{4′} is selected from i) phenyl optionally substituted with one or two groups independently selected from halogen, cyano, and trifluoromethyl, ii) heteroaryl and substituted heteroaryl selected from benzothiazole, halo-substituted benzothiazole, isoxazole, phenyl substituted isoxazole, 1,2,4-oxadiazole, phenyl substituted 1,2,4-oxadiazole, thiazole, phenyl substituted thiazole, C₁-C₄ alkyl substituted thiazole, di(C₁-C₄)alkyl substituted thiazole, 1,3,4-oxadiazole, and phenyl substituted 1,3,4-oxadiazole.

Representative compounds of the instant invention include:
N-({4-hydroxy-2-oxo-1-[4-(trifluoromethyl)benzyl]-1,2-dihydro-1,8-naphthyridin-3-yl}carbonyl)glycine;
N-{[1-(4-chlorobenzyl)-4-hydroxy-2-oxo-1,2-dihydro-1,8-naphthyridin-3-yl]carbonyl}glycine;
N-{[1-(4-bromobenzyl)-4-hydroxy-2-oxo-1,2-dihydro-1,8-naphthyridin-3-yl]carbonyl}glycine;
N-{[1-(4-cyanobenzyl)-4-hydroxy-2-oxo-1,2-dihydro-1,8-naphthyridin-3-yl]carbonyl}glycine;
N-{[1-(4-methylbenzyl)-4-hydroxy-2-oxo-1,2-dihydro-1,8-naphthyridin-3-yl]carbonyl}glycine;
N-{[1-(2-fluoro-4-trifluoromethylbenzyl)-4-hydroxy-2-oxo-1,2-dihydro-1,8-naphthyridin-3-yl]carbonyl}glycine;
N-({4-hydroxy-7-methoxy-2-oxo-1-[4-(trifluoromethyl)benzyl)-1,2-dihydro-1,8-naphthyridin-3-yl}carbonyl)glycine;
N-{[1-(1,3-benzothiazol-2-ylmethyl)-4-hydroxy-2-oxo-1,2-dihydro-1,8-naphthyridin-3-yl]carbonyl}glycine;
N-({4-hydroxy-6-iodo-2-oxo-1-[4-(trifluoromethyl)benzyl]-1,2-dihydro-1,8-naphthyridin-3-yl}carbonyl)glycine;
N-({6-cyano-4-hydroxy-2-oxo-1-[4-(trifluoromethyl)benzyl]-1,2-dihydroquinolin-3-yl}carbonyl)glycine;
N-({4-Hydroxy-2-oxo-1-[4-(trifluoromethyl)benzyl]-1,2-dihydro-1,8-naphthyridin-3-yl}carbonyl)-L-alanine;
N-({4-Hydroxy-2-oxo-1-[4-(trifluoromethyl)benzyl]-1,2-dihydro-1,8-naphthyridin-3-yl}carbonyl)-D-alanine;
N-({1-[4-(Aminocarbonyl)benzyl]-4-hydroxy-2-oxo-1,2-dihydro-1,8-naphthyridin-3-yl}carbonyl)glycine;
N-({1-[4-(trifluoromethyl)benzyl]-4-hydroxy-2-oxo-1,2-dihydro-1,8-naphthyridin-3-yl}carbonyl)-L-serine;
N-({1-[4-(trifluoromethyl)benzyl]-4-hydroxy-2-oxo-1,2-dihydro-1,8-naphthyridin-3-yl}carbonyl)-L-aspartic acid;
(2S)-2-[({4-Hydroxy-2-oxo-1-[4-(trifluoromethyl)benzyl]-1,2-dihydro-1,8-naphthyridin-3-yl}carbonyl)amino]butanoic acid;
N-[(4-Hydroxy-2-oxo-1-prop-2-yn-1-yl-1,2-dihydro-1,8-naphthyridin-3-yl)carbonyl]glycine;
N-({4-Hydroxy-2-oxo-1-[(3-phenylisoxazol-5-yl)methyl]-1,2-dihydro-1,8-naphthyridin-3-yl}carbonyl)glycine;
N-({4-Hydroxy-2-oxo-1-[(5-phenyl-1,2,4-oxadiazol-3-yl)methyl]-1,2-dihydro-1,8-naphthyridin-3-yl}carbonyl)glycine;
N-({4-Hydroxy-2-oxo-1-[(3-phenyl-1,2,4-oxadiazol-5-yl)methyl]-1,2-dihydro-1,8-naphthyridin-3-yl}carbonyl)glycine;
N-({4-Hydroxy-2-oxo-1-[(4-phenyl-1,3-thiazol-2-yl)methyl]-1,2-dihydro-1,8-naphthyridin-3-yl}carbonyl)glycine;
N-{[1-(2-Ethoxy-2-oxoethyl)-4-hydroxy-2-oxo-1,2-dihydro-1,8-naphthyridin-3-yl]carbonyl}glycine;
N-({1-[(5-Chloro-1,3-benzothiazol-2-yl)methyl]-4-hydroxy-2-oxo-1,2-dihydro-1,8-naphthyridin-3-yl}carbonyl)glycine;
N-({1-[(4-tert-Butyl-1,3-thiazol-2-yl)methyl]-4-hydroxy-2-oxo-1,2-dihydro-1,8-naphthyridin-3-yl}carbonyl)glycine;
N-({1-[(4,5-Dimethyl-1,3-thiazol-2-yl)methyl]-4-hydroxy-2-oxo-1,2-dihydro-1,8-naphthyridin-3-yl}carbonyl)glycine;
N-({4-Hydroxy-2-oxo-1-[(5-phenyl-1,3,4-oxadiazol-2-yl)methyl]-1,2-dihydro-1,8-naphthyridin-3-yl}carbonyl)glycine;
{[(4-Hydroxy-2-oxo-1-{[6-(trifluoromethyl)pyridin-3-yl]methyl}-1,2-dihydro-1,8-naphthyridin-3-yl]carbonyl}amino)acetic acid;
{[(6-Chloro-4-hydroxy-2-oxo-1-{[6-(trifluoromethyl)pyridin-3-yl]methyl}-1,2-dihydro-1,8 naphthyridin-3-yl)carbonyl]amino}acetic acid;
(2S)-2-({[1-(1,3-Benzothiazol-2-yl methyl)-4-hydroxy-2-oxo-1,2-dihydro-1,8-naphthyridin-3-yl]carbonyl}amino)propanoic acid;
(2S)-2-[({4-Hydroxy-2-oxo-1-[4-(trifluoromethyl)benzyl]-1,2-dihydro-1,8-naphthyridin-3-yl}carbonyl)amino]succinic acid;
({[1-(1,3-Benzothiazol-2-yl methyl)-4-hydroxy-6-iodo-2-oxo-1,2-dihydro-1,8-naphthyridin-3-yl]carbonyl}amino)acetic acid;
2-[({4-Hydroxy-2-oxo-1-[4-(trifluoromethyl)benzyl]-1,2-dihydro-1,8-naphthyridin-3-yl}carbonyl)amino]-2-methylpropanoic acid;
(2S)-2-[9{4-Hydroxy-2-oxo-1-[4-(trifluoromethyl)benzyl]-1,2-dihydro-1,8-naphthyridin-3-yl}carbonyl]amino]butanoic acid;
(2S)-2-[({1-[(5-Chloro-1,3-benzothiazol-2-yl)methyl]-4-hydroxy-2-oxo-1,2-dihydro-1,8-naphthyridin-3-yl}carbonyl)amino]propanoic acid;
(2R)-2-[({4-Hydroxy-2-oxo-1-[4-(trifluoromethyl)benzyl]-1,2-dihydro-1,8-naphthyridin-3-yl}carbonyl)amino]succinic acid;
(2S)-2-({[1-(1,3-Benzothiazol-2-ylmethyl)-4-hydroxy-2-oxo-dihydro-1,8-naphthyridin-3-yl]carbonyl}amino)succinic acid;
(2S)-2-{[(4-Hydroxy-2-oxo-1-{[6-(trifluoromethyl)pyridin-3-yl]methyl}-1,2-dihydro-1,8-naphthyridin-3-yl)carbonyl]amino}propanoic acid;
(2S)-2-[({1-[2-Fluoro-4-(trifluoromethyl)benzyl]-4-hydroxy-2-oxo-1,2-dihydro-1,8-naphthyridin-3-yl}carbonyl)amino]propanoic acid; and
({[1-(1,3-Benzothiazol-2-ylmethyl)-6-chloro-4-hydroxy-2-oxo-1,2-dihydro-1,8-naphthyridin-3-yl]carbonyl}amino)acetic acid; or a pharmaceutically acceptable salt or solvate thereof.

As used herein, unless specified otherwise, “alkyl” includes both branched- and straight-chain saturated aliphatic hydrocarbon groups, including all isomers, having the specified number of carbon atoms; for example, “C₁-C₆ alkyl” (or “C₁-C₆ alkyl”) includes all of the hexyl alkyl and pentyl alkyl isomers as well as n-, iso-, sec- and t-butyl, n- and isopropyl, ethyl and methyl. “Alkylene” refers to both branched- and straight-chain saturated aliphatic hydrocarbon groups, including all isomers, having the specified number of carbons, and having two terminal end chain attachments; for example, the term “A-C₄alkylene-B” represents, for example, A-CH₂—CH₂—CH₂—CH₂—B, A-CH₂—CH₂—CH(CH₃)—CH₂—B, A-CH₂—CH(CH₂CH₃)—B, A-CH₂—C(CH₃)(CH₃)—B, and the like. “Alkoxy” represents a linear or branched alkyl group of indicated number of carbon atoms attached through an oxygen bridge; for example “C₁-C₆ alkoxy” includes —OCH₃, —OCH₂CH₃, —OCH(CH₃)₂, —O(CH₂)₅CH₃, and the like.

Unless otherwise specifically noted as only unsubstituted or only substituted, alkyl and alkoxy groups are unsubstituted or substituted with 1 to 3 substituents on one or more carbon atoms (also referred to as “optionally substituted). Unless the substituents are specifically provided, substituents for substituted or optionally substituted alkyl and alkoxy are independently selected from halo, NH₂, N(C₁-C₆ alkyl)₂, NO₂, oxo, CN, N₃, —OH, —O(C₁-C₆ alkyl), C₃-C₁₀ cycloalkyl, C₂-C₆ alkenyl, C₂-C₆ alkynyl, (C₀-C₆ alkyl)S(O)₀₋₂(C₀-C₆ alkylene)-, (C₀-C₆ alkyl)C(O)NH—, H₂N—C(NH)—, —O(C₁-C₆ alkyl)CF₃, (C₀-C₆ alkyl)C(O)—, (C₀-C₆ alkyl)OC(O)—, (C₀-C₆ alkyl)O(C₁-C₆ alkylene)-, (C₀-C₆ alkyl)C(O)₁₋₂(C₀-C₆ alkylene)-, (C₀-C₆ alkyl)OC(O)NH—, —NH(C₁-C₆ alkylene)NHC(O)NH(C₁-C₆ alkyl), —NHC(O)OC₁-C₆ alkyl, —NH(C₁-C₆ alkylene)NHSO₂(C₁-C₆ alkyl), —(C₀-C₆ alkylene)NHSO₂(C₁-C₆ alkyl), aryl, aralkyl, heterocycle, and heterocyclylalkyl, wherein aryl, aralkyl, heterocycle, and heterocyclylalkyl are optionally substituted with 1 to 3 groups independently selected from halogen and cyano.

The term “C₃₋₁₀ cycloalkyl” (or “C₃-C₁₀ cycloalkyl”) means a cyclic ring of an alkane having three to ten total ring carbon atoms (i.e., cyclopropyl, cyclobutyl, cyclopentyl, cyclohexyl, cycloheptyl, cyclooctyl, cyclononyl or cyclodecyl).

The term “C₂₋₁₀ alkenyl” (or “C₂-C₁₀ alkenyl”) means a straight or branched two to ten carbon chain with at least one carbon-carbon double bond. Examples of alkenyl include, but are not limited to, vinyl, allyl, isopropenyl, pentenyl, hexenyl, heptenyl, 1-propenyl, 2-butenyl, 2-methyl-2-butenyl, 2,4-hexadienyl, and the like. The term “C₅-C₁₀ cycloalkenyl” (or “C₅-C₁₀ cycloalkenyl”) means a non-aromatic monocyclic ring having from 5 to 10 carbon atoms in the ring with at least one carbon-carbon double bond.

The term “C₂₋₁₀ alkynyl” (or “C₂-C₁₀ alkynyl”) means a straight or branched two to ten carbon chain with at least one carbon-carbon triple bond. Examples of alkynyl include, but are not limited to ethynyl, propargyl, 1-propynyl, 2-butynyl, and the like.

The term “aryl” refers to aromatic mono- and poly-carbocyclic ring systems, wherein the individual carbocyclic rings in the polyring systems are fused or attached to each other via a single bond. Suitable aryl groups include phenyl, naphthyl, and biphenyl.

The term “heteroaryl” (or heteroaromatic) refers to a 5- or 6-membered monocyclic aromatic ring or a 7- to 12-membered bicyclic aromatic ring which consists of carbon atoms and one or more heteroatoms selected from N, O and S. In the case of substituted heteroaryl rings containing at least one nitrogen atom (e.g., pyridine) or at least one sulfur atom (e.g., thiophene), such substitutions can be those resulting in N-oxide or S-oxide (including S-dioxide) formation. Representative examples of monocyclic heteroaromatic rings include pyridyl, pyrrolyl, pyrazinyl, pyrimidinyl, pyridazinyl, thienyl (or thiophenyl), furanyl, imidazolyl, pyrazolyl, triazolyl, tetrazolyl, oxazolyl, isooxazolyl, oxadiazolyl, thiazolyl, isothiazolyl, and thiadiazolyl. Representative examples of bicyclic heteroaromatic rings include benzotriazolyl, indolyl, isoindolyl, indazolyl, quinoxalinyl, quinazolinyl, cinnolinyl, chromanyl, isochromanyl, quinolinyl, isoquinolinyl, benzofuranyl, imidazo(2,1-b)(1,3)thiazole, (i.e.,

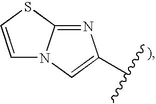

benzothienyl, benzimidazolyl, benzothiazolyl, and benzoxazolyl.

Unless otherwise specifically noted as only unsubstituted or only substituted, cycloalkyl, cycloalkenyl, cycloalkyl, aryl (including phenyl) and heteroaryl groups are unsubstituted or substituted (also referred to as “optionally substituted”). Unless the substituents are specifically provided, substituents for substituted or optionally substituted cycloalkyl, cycloalkenyl, aryl (including phenyl, and as an isolated substituent or as part of a substituent such as in aryloxy and aralkyl), heteroaryl (as an isolated substituent or as part of a substituent such as in heteroaryloxy and heteroaralkyl) are one to three groups independently selected from halo, C₁-C₆ alkyl optionally substituted with one to five fluorine, NH₂, N(C₁-C₆ alkyl)₂, NO₂, oxo, CN, N₃, —OH, —0(C₁-C₆ alkyl) optionally substituted with one to five fluorine, C₃-C₁₀ cycloalkyl, C₂-C₆ alkenyl, C₂-C₆ alkynyl, (C₀-C₆ alkyl)S(O)₀₋₂—, aryl-S(O)₀₋₂—, (C₀-C₆ alkyl)S(O)₀₋₂(C₀-C₆ alkylene)-, (C₀-C₆ alkyl)C(O)NH—, H₂N—C(NH)—, (C₀-C₆ alkyl)C(O)—, (C₀-C₆ alkyl)OC(O)—, (C₀-C₆alkyl)O(C₁-C₆ alkylene)-, (C₀-C₆ alkyl)C(O)₁₋₂(C₀-C₆ alkylene)-, (C₀-C₆ alkyl)₂NC(O)—, (C₀-C₆ alkyl)OC(O)NH—, aryl, aralkyl, heteroaryl, heteroaralkyl, halo-aryl, halo-aralkyl, halo-heteroaryl, halo-heteroaralkyl, cyano-aryl, cyano-aralkyl, cyano-heteroaryl and cyano-heteroaralkyl.

The term “halogen” (or “halo”) refers to fluorine, chlorine, bromine and iodine (alternatively referred to as fluoro (F), chloro (Cl), bromo (Br), and iodo (I)).

The term “C₀” as employed in expressions such as “C₀₋₆ alkylene” means a direct covalent bond; or when employed in expressions such as “C₀₋₆ alkyl” means hydrogen. Similarly, when an integer defining the presence of a certain number of atoms in a group is equal to zero, it means that the toms adjacent thereto are connected directly by a bond; for example, in the structure

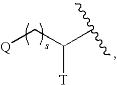

wherein s is an integer equal to zero, 1 or 2, the structure is

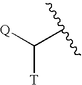

when s is zero; or it means that the indicated atom is absent; for example —S(O)₀— means —S—.

Unless expressly stated to the contrary, an “unsaturated” ring is a partially or fully unsaturated ring. For example, an “unsaturated monocyclic C₆ carbocycle” refers to cyclohexene, cyclohexadiene, and benzene.

Unless expressly stated to the contrary, all ranges cited herein are inclusive. For example, a heterocycle described as containing from “1 to 4 heteroatoms” means the heterocycle can contain 1, 2, 3 or 4 heteroatoms.

When any variable occurs more than one time in any constituent or in any formula depicting and describing compounds of the invention, its definition on each occurrence is independent of its definition at every other occurrence. Also, combinations of substituents and/or variables are permissible only if such combinations result in stable compounds. For variable definitions containing terms having repeated terms, e.g., (CRⁱRⁱ)ᵣ, where r is the integer 2, Rⁱ is a defined variable, and R^{j} is a defined variable, the value of Rⁱ may differ in each instance in which it occurs, and the value of R^{j} may differ in each instance in which it occurs. For example, if Rⁱ and R^{j} are independently selected from the group consisting of methyl, ethyl, propyl and butyl, then (CRⁱR^{j})₂ can be

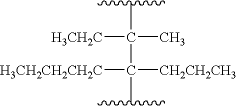

### Optical Isomers-Diastereomers-Geometric Isomers-Tautomers

Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The above Formula I is shown without a definitive stereochemistry at certain positions. The present invention includes all stereoisomers of Formula I and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general Formula I may be obtained by stereospecific synthesis using optically pure starting materials or reagents of known configuration.

When compounds described herein contain olefinic double bonds, unless specified otherwise, such double bonds are meant to include both E and Z geometric isomers.

Some of the compounds described herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are included within the scope of the present invention.

### Salts

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from pharmaceutically acceptable organic non-toxic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

### Solvates

The present invention includes within its scope solvates of compounds of Formula I. As used herein, the term “solvate” refers to a complex of variable stoichiometry formed by a solute (i.e., a compound of Formula I) or a pharmaceutically acceptable salt thereof and a solvent that does not interfere with the biological activity of the solute. Examples of solvents include, but are not limited to water, ethanol, and acetic acid. When the solvent is water, the solvate is known as hydrate; hydrate includes, but is not limited to, hemi-, mono, sesqui-, di- and trihydrates.

### Prodrugs

The present invention includes within its scope the use prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with a compound of formula I or with a compound which may not be a compound of formula I, but which converts to a compound of formula I in vivo after administration to the patient. Conventional procedures for the selection and preparation of suitable prodrug derivatives are described, for example, in “Design of Prodrugs,” ed. H. Bundgaard, Elsevier, 1985.

### Utilities

Compounds of the present invention are inhibitors of hypoxia-inducible factor (HIF) prolyl hydroxylases, and as such are useful in the treatment and prevention of diseases and conditions in which HIF modulation is desirable, such as anemia and ischemia. Compounds of the invention can be used in a selective and controlled manner to induce hypoxia-inducible factor stabilization and to rapidly and reversibly stimulate erythropoietin production and secretion. Accordingly, another aspect of the present invention provides a method of treating or preventing a disease or condition in a mammal, the treatment or prevention of which is effected or facilitated by HIF prolyl hydroxylase inhibition, which comprises administering an amount of a compound of Formula I that is effective for inhibiting HIF prolyl hydroxylase. This aspect of the present invention further includes the use of a compound of Formula I in the manufacture of a medicament for the treatment or prevention of a disease or condition modulated by HIF prolyl hydroxylase.

In one embodiment is a method of enhancing endogenous production of erythropoietin in a mammal which comprises administering to said mammal an amount of a compound of Formula I that is effective for enhancing endogenous production of erythropoietin.

Another embodiment is a method of treating anemia in a mammal which comprises administering to said mammal a therapeutically effective amount of a compound of Formula I. “Anemia” includes, but is not limited to, chronic kidney disease anemia, chemotherapy-induced anemia (e.g., anemia resulting from antiviral drug regimens for infectious diseases, such as HIV and hepatitis C virus), anemia of chronic disease, anemia associated with cancer conditions, anemia resulting from radiation treatment for cancer, anemias of chronic immune disorders such as rheumatoid arthritis, inflammatory bowel disease, and lupus, and anemias due to menstruation or of senescence or in other individuals with iron processing deficiencies such as those who are iron-replete but unable to utilize iron properly.

Another embodiment is a method of treating ischemic diseases in a mammal, which comprises administering to said mammal a therapeutically effective amount of a compound of Formula I.

### Combination Therapy

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I.

### Route of Administration/Dosage

The compounds of this invention can be administered for the treatment or prevention of afflictions, diseases and illnesses according to the invention by any means that effects contact of the active ingredient compound with the site of action in the body of a warm-blooded animal. For example, administration can be oral, topical, including transdermal, ocular, buccal, intranasal, inhalation, intravaginal, rectal, intracisternal and parenteral. The term “parenteral” as used herein refers to modes of administration which include subcutaneous, intravenous, intramuscular, intraarticular injection or infusion, intrasternal and intraperitoneal. For the purpose of this disclosure, a warm-blooded animal is a member of the animal kingdom possessed of a homeostatic mechanism and includes mammals and birds.

The compounds can be administered by any conventional means available for use in conjunction with pharmaceuticals, either as individual therapeutic agents or in a combination of therapeutic agents. They can be administered alone, but are generally administered with a pharmaceutical carrier selected on the basis of the chosen route of administration and standard pharmaceutical practice.

The dosage administered will be dependent on the age, health and weight of the recipient, the extent of disease, kind of concurrent treatment, if any, frequency of treatment and the nature of the effect desired. Usually, a daily dosage of active ingredient compound will be from about 1.0-2000 milligrams per day. Ordinarily, from 10 to 500 milligrams per day in one or more applications is effective to obtain desired results. These dosages are the effective amounts for the treatment and prevention of afflictions, diseases and illnesses described above, e.g., anemia.

### Pharmaceutical Composition

Another aspect of the present invention provides pharmaceutical compositions which comprises a compound of Formula I and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

The pharmaceutical compositions of the present invention comprise a compound represented by Formula I (or pharmaceutically acceptable salts thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The active ingredient can be administered orally in solid dosage forms, such as capsules, tablets, troches, dragées, granules and powders, or in liquid dosage forms, such as elixirs, syrups, emulsions, dispersions, and suspensions. The active ingredient can also be administered parenterally, in sterile liquid dosage forms, such as dispersions, suspensions or solutions. Other dosages forms that can also be used to administer the active ingredient as an ointment, cream, drops, transdermal patch or powder for topical administration, as an ophthalmic solution or suspension formation, i.e., eye drops, for ocular administration, as an aerosol spray or powder composition for inhalation or intranasal administration, or as a cream, ointment, spray or suppository for rectal or vaginal administration.

Gelatin capsules contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propylparaben, and chlorobutanol.

Suitable pharmaceutical carriers are described in Remington's Pharmaceutical Sciences, A. Osol, a standard reference text in this field.

For administration by inhalation, the compounds of the present invention may be conveniently delivered in the form of an aerosol spray presentation from pressurized packs or nebulisers. The compounds may also be delivered as powders which may be formulated and the powder composition may be inhaled with the aid of an insufflation powder inhaler device. The preferred delivery system for inhalation is a metered dose inhalation (MDI) aerosol, which may be formulated as a suspension or solution of a compound of Formula I in suitable propellants, such as fluorocarbons or hydrocarbons.

For ocular administration, an ophthalmic preparation may be formulated with an appropriate weight percent solution or suspension of the compounds of Formula I in an appropriate ophthalmic vehicle, such that the compound is maintained in contact with the ocular surface for a sufficient time period to allow the compound to penetrate the corneal and internal regions of the eye.

Useful pharmaceutical dosage-forms for administration of the compounds of this invention include, but are not limited to, hard and soft gelatin capsules, tablets, parenteral injectables, and oral suspensions.

A large number of unit capsules are prepared by filling standard two-piece hard gelatin capsules each with 100 milligrams of powdered active ingredient, 150 milligrams of lactose, 50 milligrams of cellulose, and 6 milligrams magnesium stearate.

A mixture of active ingredient in a digestible oil such as soybean oil, cottonseed oil or olive oil is prepared and injected by means of a positive displacement pump into gelatin to form soft gelatin capsules containing 100 milligrams of the active ingredient. The capsules are washed and dried.

A large number of tablets are prepared by conventional procedures so that the dosage unit is 100 milligrams of active ingredient, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

A parenteral composition suitable for administration by injection is prepared by stirring 1.5% by weight of active ingredient in 10% by volume propylene glycol. The solution is made to volume with water for injection and sterilized.

An aqueous suspension is prepared for oral administration so that each 5 milliliters contain 100 milligrams of finely divided active ingredient, 100 milligrams of sodium carboxymethyl cellulose, 5 milligrams of sodium benzoate, 1.0 grams of sorbitol solution, U.S.P., and 0.025 milliliters of vanillin.

The same dosage forms can generally be used when the compounds of this invention are administered stepwise or in conjunction with another therapeutic agent. When drugs are administered in physical combination, the dosage form and administration route should be selected depending on the compatibility of the combined drugs. Thus the term coadministration is understood to include the administration of the two agents concomitantly or sequentially, or alternatively as a fixed dose combination of the two active components.

### Synthesis

Methods for preparing the compounds of this invention are illustrated in the following schemes. Other synthetic protocols will be readily apparent to those skilled in the art. The examples illustrate the preparation of the compounds of Formula I and as such are not to be considered as limiting the invention set forth in the claims appended hereto. Unless otherwise indicated, all variables are as previously defined.

Intermediates useful for the preparation of the compounds in the present invention are known in the art or may be prepared using chemical methodologies known to those skilled in the art. Examples of reported intermediates and methods for their preparation include ethyl 1-benzyl-4-hydroxy-2-oxo-1,2-dihydro-1,8-naphthyridine-3-carboxylate (IIa) and related substituted analogs (IIb) (see WO 2005/021546); ethyl 1-alkyl-4-hydroxy-7-methyl-2-oxo-1,2-dihydro-1,8-naphthyridine-3-carboxylate analogs (He) (see Kuroda, et. al. in Journal of Medicinal Chemistry, 1992, 35, 1130-1136); ethyl 1-substituted-4-hydroxy-2-oxo-1,2-dihydro-1,8-naphthyridine-3-carboxylate analogs (IId) (see Kuroda, et. al. in Bioorganic & Medicinal Chemistry Letters, 2005, 15, 1577-1582); and ethyl 4-hydroxy-2-oxo-6-pyridin-4-yl-1,2-dihydro-1,8-naphthyridine-3-carboxylate (IIe) and its 8-oxide (IIf) (see Haber, et. al. in Journal fur Praktische Chemie, 1991, 333, 637-642). Other general methods that are applicable to the preparation of intermediates H can be found in Sherlock, et. al. in Journal of Medicinal Chemistry, 1988, 31, 2108-2121.

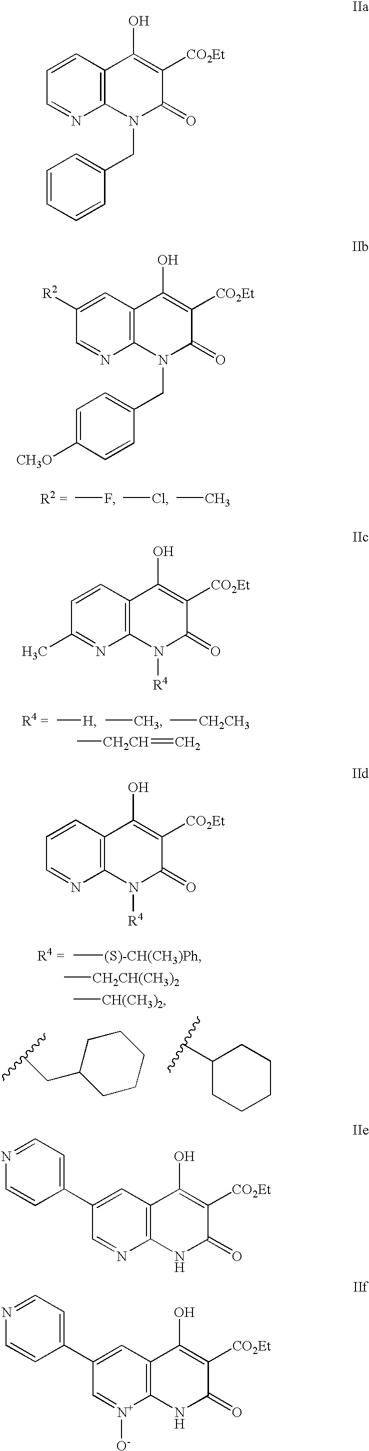

The compounds in the present invention can be prepared using methods illustrated in the following schemes. In Scheme 1, an alkyl ester of 4-hydroxy-2-oxo-1,8-naphthyridin-3-carboxylate and an α- or β-amino acid ester (III) can be combined in a suitable solvent (e.g., toluene, xylenes, bromobenzene, N,N-dimethylformamide, N,N-dimethylacetamide, dimethoxymethane, N-methyl-pyrrolidinone, dimethylsulfoxide, ethanol, 2-propanol, butanol) and the resulting mixture heated to give intermediate IV. An example for synthesizing intermediates IV would be heating II with 1-2 molar equivalents of III, where n=1 and R⁷=—C(CH₃)₃, in ethanol/dimethoxymethane at 130° C. for 1 to 3 hours. General methods to cleave esters are applicable for converting ester IV to carboxylic acid I (R⁷=—H) and references for these can be found in the literature (e.g., Greene and Wuts, Eds. Protective Groups in Organic Synthesis, 3^{rd} Edition, Wiley-Interscience, 1999). An example for effecting the conversion of IV, where (R⁷=—C(CH₃)₃) to I (R⁷=—H), is treating IV with an acid in a solvent (e.g. 6 M HCl in water, trifluoroacetic acid in CH₂Cl₂) and stirring the resulting mixture at room temperature for 12-24 hours.

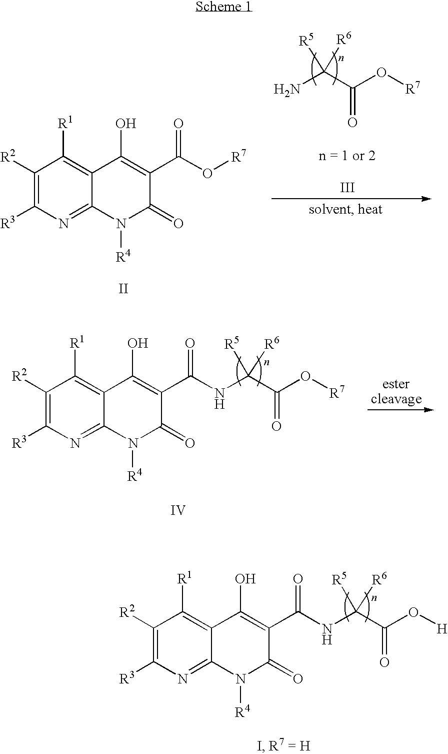

Several methods to prepare II are shown in Scheme 2. In one method 2H-pyrido[2,3-d][1,3]oxazine-2,4(1H)-dione of the general structure VI or VII is treated with a dialkylmalonate and a strong base (i.e., NaH, KH) neat or in an appropriate solvent (e.g., N,N-dimethylformamide, N,N-dimethylacetamide, N-methylpyrrolidinone) at or above ambient temperature for 2-18 hours. The dione VI, in turn, may be prepared by a variety of methods. In one method, a substituted 2-amino nicotinic acid (V) was treated with phosgene or an equivalent chemical reagent (e.g., diphosgene, triphosgene, N,N′-carbonyldiimidazole, ethyl chloroformate) in a suitable solvent (e.g., CH₂Cl₂, THF, 1,4-dioxane, toluene, xylenes) at or above room temperature to give intermediate VI. Another method for synthesizing intermediates VI involves heating an equimolar amount of a substituted 2-carboxmido nicotinic acid (Va) in the presence of lead tetraaceate in DMF at 55° C. for an hour (see U.S. Pat. No. 3,947,442).

The dione VII can be obtained from VI when the latter is treated with an alkyl halide or an alkyl trifluoromethylsulfonic acid ester in the presence of a base (e.g., triethylamine, diisopropylethylamine, Na₂CO₃, NaH) in an appropriate solvent (e.g., toluene, xylenes, bromobenzene, N,N-dimethylformamide, N,N-dimethylacetamide, N-methylpyrrolidinone, dimethylsulfoxide) to give VII (R₄=alkyl). Alternative methods for synthesizing VII from VI include, but are not limited to, the Mitsunobu reaction (treatment of VI and the requisite alcohol with a trialkylphosphine and an dialkylazodicarboxylate in an appropriate solvent as described by Coppola, G. M. et. al. (Synth. Comm., 2002, 32, 1009-1013), or substitution of an 2-N—(R4-amino)- or 2-(N—R4-amino)nicotinic acid for V in the reactions described above.

Another method to prepare H (where R₄≠—H) utilizes 2-chloronicotinic acid esters of the general structure VIII as the starting material. Displacement of the 2-Cl can be accomplished by heating VIII with the appropriate amine with or without an added base (e.g., triethylamine, diisopropylethylamine, pyridine, DBU) to give a nicotinate ester of the general structure IX. Acylation of IX with an alkyl malonyl chloride gives an amide X. Ring closure to give II can be carried out by treating X with a base (e.g., sodium methoxide, sodium bis(trimethylsilyl) amide, potassium t-butoxide, sodium hydride) in a suitable solvent (1,2-dimethoxyethane, THF, toluene). Ring closure to give II can also be carried out by treating IX with diethylmalonate and a base (e.g., sodium methoxide, sodium bis(trimethylsilyl)amide, potassium t-butoxide, sodium hydride) in a suitable solvent (1,2-dimethoxyethane, THF, toluene).

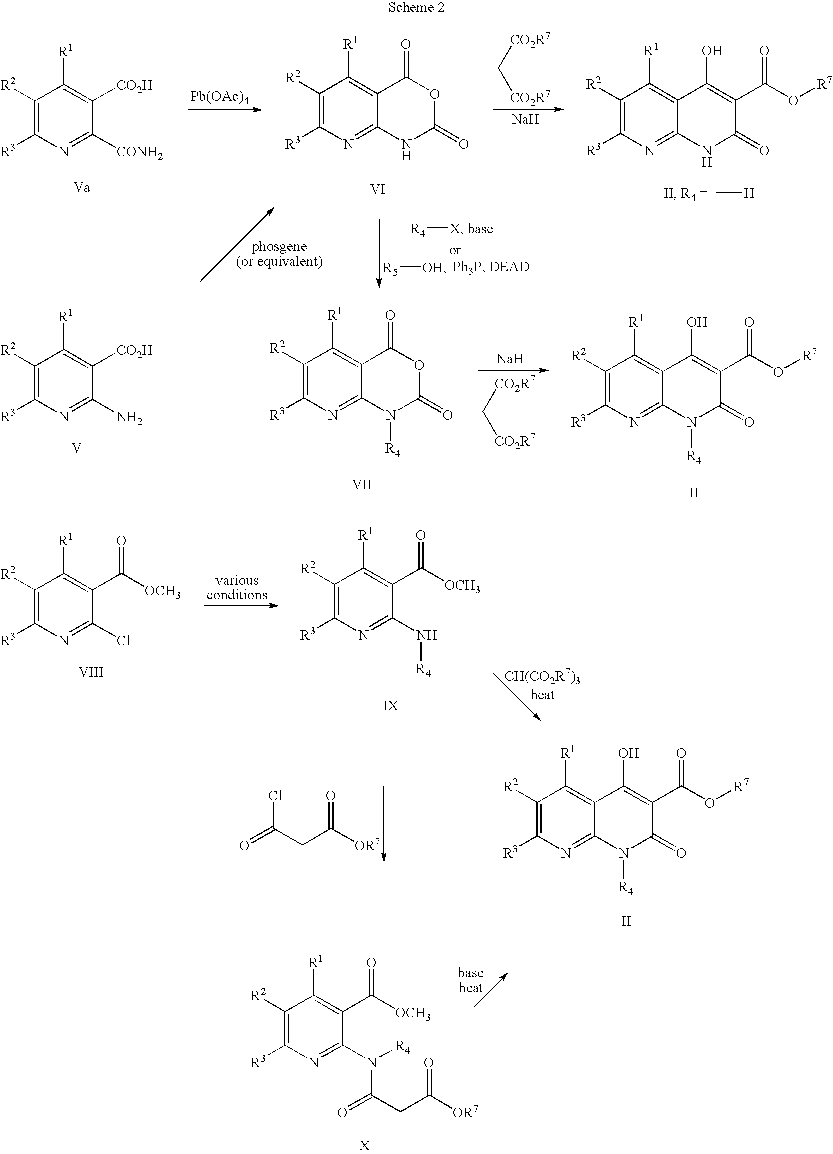

There may be cases where R¹-R⁶ of I contain one or more asymmetric centers. When this occurs, the individual stereoisomers of I can be obtained by methods known to those skilled in the art which include (but are not limited to): stereospecific synthesis, resolution of salts of I or any of the intermediates used in its preparation with enantiopure acids or bases, resolution of I or any of the intermediates used in its preparation by HPLC employing enantiopure stationary phases.

### Biological Assays

The biological activity of the present compounds may be evaluated using assays described herein below:

To each well of a 96-well plate was added 14 of test compound in DMSO and 20 μl of assay buffer (50 mM Tris pH 7.4/0.01% Tween-20/0.1 mg/ml bovine serum albumin/10 μM ferrous sulfate/1 mM sodium ascorbate/20 μg/ml catalase) containing 0.15 μg/ml FLAG-tagged full length PHD2 expressed in and purified from baculovirus-infected Sf9 cells. After a 30 mM preincubation at room temperature, the enzymatic reactions were initiated by the addition of 4 μL of substrates (final concentrations of 0.2 μM 2-oxoglutarate and 0.5 μM HIF-1α peptide biotinyl-DLDLEMLAPYIPMDDDFQL). After 2 hr at room temperature, the reactions were terminated and signals were developed by the addition of a 25 μL quench/detection mix to a final concentration of 1 mM ortho-phenanthroline, 0.1 mM EDTA, 0.5 nM anti-(His)₆ LANCE reagent (Perkin-Elmer Life Sciences), 100 nM AF647-labeled streptavidin (Invitrogen), and 2 μg/ml (His)₆-VHL complex (S. Tan (2001) Protein Expr. Purif. 21, 224-234). The ratio of time resolved fluorescence signals at 665 and 620 nm was determined, and percent inhibition was calculated relative to an uninhibited control sample run in parallel.

| EXAMPLE | IC₅₀ (nM) |
|---|---|
| 1 | 2.1 |
| 2 | 1.6 |
| 3 | 1.9 |
| 4 | 1.6 |
| 5 | 1.8 |
| 6 | 1.6 |
| 7 | 1.5 |
| 8 | 0.7 |
| 9 | 1.3 |
| 10 | 1.2 |
| 11 | 6.0 |
| 12 | 1100 |
| 13 | 1.2 |
| 14 | 160 |
| 15 | 25 |
| 17 | 160 |
| 18 | 21 |
| 19 | 0.4 |
| 20 | 0.9 |
| 21 | 0.8 |
| 22 | 0.8 |
| 23 | 8.5 |
| 24 | 1.1 |
| 25 | 2.0 |
| 26 | 1.3 |
| 27 | 2.6 |
| 28 | 0.85 |
| 29 | 0.65 |
| 30 | 2.7 |
| 31 | 25 |
| 34 | 160 |
| 35 | 5.9 |
| 38 | 2.7 |
| 39 | 8.2 |
| 40 | 1.5 |

Inhibition of the catalytic activity of HIF-PHD1 and HIF-PHD3 can be determined similarly.

The following examples are provided to illustrate the invention only and are not to be construed as limiting the scope of the invention in any way.

### Example 1

### N-({4-Hydroxy-2-oxo-1-[4-(trifluoromethyl)benzyl]-1,2-dihydro-1,8-naphthyridin-3-yl}carbonyl)glycine

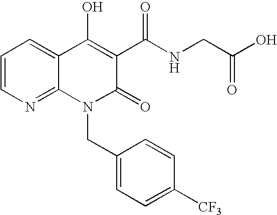

### Step A: 2H-Pyrido[2,3-d][1,3]oxazine-2,4(1H)-dione

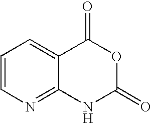

To a solution of 2-(carbamoyl)nicotinic acid (3.2 g, 19.26 mmol) in DMF (30 mL) at 0° C. was added lead tetraacetate (8.5, 19.26 mmol) in small portions. The resulting solution was stirred and allowed to warm to ambient temperature. After heating the reaction mixture at 55° C. for an hour, it was quenched with water (30 mL). The precipitate that was formed was filtered, washed with water, and dried to give 2.72 g of the title compound as a solid: ¹H NMR (500 MHz, DMSO-d₆) δ 12.27 (s), 8.65 (d, 1H, J=4.3 Hz), 8.29 (d, 1H, J=7.5 Hz), 7.31 (d, 1H, J=7.3 Hz).

### Step B: 1-[4-(Trifluoromethyl)benzyl]-2H-pyrido[2,3-d][1,3]oxazine-2,4(1H)-dione

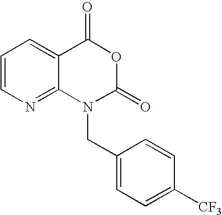

To a solution of 5.0 g (30.5 mmol) of the compound of Step A in 50 mL of dimethylacetamide was added 1.46 g (36.6 mmol, 60% wt. dispersion in mineral oil) sodium hydride at ambient temperature. The mixture was stirred at 60° C. for an additional 20 min and was then cooled to 0° C. The mixture was treated with a solution of 4-(trifluoromethyl)benzyl bromide (8.74 g, 36.6 mmol) in DMF (10 mL) and then warmed to ambient temperature. After 4 h, the DMF from the reaction mixture was removed under vacuum and the residue was diluted with a mixture of ice and water. The solid precipitate was filtered, washed with water and dried to afford 8.7 g the desired product which was used without further purification: ¹H NMR (500 MHz, DMSO-d₆) δ 8.71 (dd, 1H, J=4.8 and 1.9 Hz), 8.43 (dd, 1H, J=4.9 and 1.6 Hz), 7.66 (d, 2H, J=8.4 Hz), 7.63 (d, 1H, J=8.7 Hz), 7.40 (dd, 2H, J=4.8 and 4.9 Hz), 5.43 (s, 2H).

### Step C: Ethyl-4-hydroxy-2-oxo-1-[4-(trifluoromethyl)benzyl]-1,2-dihydro-1,8-naphthyridine-3-carboxylate

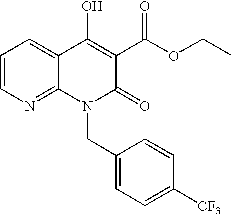

To a mixture of NaH (60% suspension in mineral oil, 1.62 g, 40.5 mmol) in dimethylformamide (75 mL) was added diethylmalonate (6.18 mL, 40.5 mmol) at 0° C. The resulting mixture was stirred at 0° C. for 30 min and ambient temperature for 20 min, then was treated with 8.7 g (27 mmol) of the compound of Step B in 10 mL of DMF. The reaction mixture was stirred at 60° C. for 3 h. The mixture was cooled, the DMF was removed under vacuum and the residue was diluted with water. The pH of the aqueous mixture was adjusted to 4-5 with 1 N HCl and then extracted with EtOAc. The organic layer was washed with saturated NaCl, dried over magnesium sulfate, treated with charcoal, filtered and concentrated to give an oil. Trituration with hexanes/ether and filtration of the solid and drying gave 5.2 g of the title compound: ¹H NMR (500 MHz, CDCl₃) δ 8.71 (dd, 1H, J=4.6 nd 1.9 Hz), 8.49 (dd, 1H, J=6.2 and 1.6 Hz) 7.57 (d, 2H, J=8.3 Hz), 7.57 (d, 2H, J=8.2 Hz), 7.40 (dd, 2H, J=4.6 and 3.2 Hz, 5.76 (s, 2H), 4.55 (q, 2H, J=7.1 Hz) and 1.51 (t, 3H, 7.0 Hz). MS: m/z 347 (M-EtOH).

### Step D: tert-Butyl N-({4-hydroxy-2-oxo-1-[4-(trifluoromethyl)benzyl]-1,2-dihydro-1,8-naphthyridin-3-yl}carbonyl)glycinate

A mixture 800 mg (2.04 mmol) of the compound of Step C and 320 mg (2.45 mmol) of tert-butylglycine in 5 mL of ethanol was stirred at 130° C. under microwave conditions for 3 h. The solution was cooled to room temperature and the white solid that precipitated was filtered. The precipitate was washed with hexanes/ether and dried to give 930 mg of the title compound as a white solid: ¹H NMR (500 MHz, DMSO-d₆) δ 10.31 (br s, 1H), 8.77 (dd, 1H, J=4.6 and 1.6 Hz), 8.50 (dd, 1H, J=6.4 and 1.6 Hz), 7.63-7.44 (m, 3H), 5.70 (s, 2H), 4.08 (d, 2H, J=5.7 Hz), 1.42 (s, 9H); MS: m/z 478 (M+H).

### Step E: N-({4-Hydroxy-2-oxo-1-[4-(trifluoromethyl)benzyl]-1,2-dihydro-1,8-naphthyridin-3-yl}carbonyl)glycine

To a solution of 4.7 g (9.8 mmol) the compound of Step D in 40 mL of dichloromethane was added 10 mL of trifluoroacetic acid (10 mL) at room temperature. The mixture was stirred at ambient temperature for 12 h. The reaction mixture was concentrated, lyophilized with toluene, triturated with hexane, filtered and dried to give 3.9 of the title compound as a white solid: ¹H NMR (500 MHz, DMSO-d₆) δ 12.98 (br s, 1H), 10.31 (t, 1H, J=5.2 Hz), 8.76 (dd, 1H, J=4.6 and 1.4 Hz), 8.49 (dd, 1H, J=6.4 and 1.3 Hz), 7.64-7.43 (m, 3H), 5.7 (s, 2H), 4.12 (d, 2H, J=5.5 Hz); MS: m/z 423 (M+H).

### Example 2

### N-{[1-(4-Chlorobenzyl)-4-hydroxy-2-oxo-1,2-dihydro-1,8-naphthyridin-3-yl]carbonyl}glycine

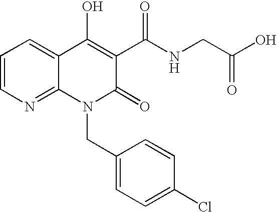

The title compound was prepared using procedures analogous to those described in EXAMPLE 1, substituting 4-(chloro)benzyl bromide for 4-(trifluoromethyl)benzyl bromide in Step B: MS: m/z 388 (M+H).

### Example 3

### N-{[1-(4-Bromobenzyl)-4-hydroxy-2-oxo-1,2-dihydro-1,8-naphthyridin-3-yl]carbonyl}glycine

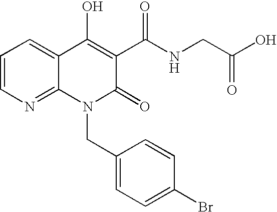

The title compound was prepared using procedures analogous to those described in EXAMPLE 1, substituting 4-(bromo)benzyl bromide for 4-(trifluoromethyl)benzyl bromide in Step B: MS: m/z 433 (M+H).

### Example 4

### N-{[1-(4-Cyanobenzyl)-4-hydroxy-2-oxo-1,2-dihydro-1,8-naphthyridin-3-yl]carbonyl}glycine

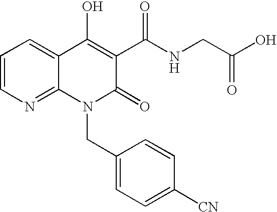

The title compound was prepared using procedures analogous to those described in EXAMPLE 1, substituting 4-(cyano)benzyl bromide for 4-(trifluoromethyl)benzyl bromide in Step B: MS: m/z 379 (M+H).

### Example 5

### N-{[1-(4-Methylbenzyl)-4-hydroxy-2-oxo-1,2-dihydro-1,8-naphthyridin-3-yl]carbonyl}glycine

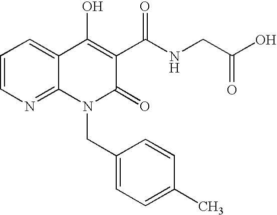

The title compound was prepared using procedures analogous to those described in EXAMPLE 1, substituting 4-(methyl)benzyl bromide for 4-(trifluoromethyl)benzyl bromide in Step B: MS: m/z 368 (M+H).

### Example 6

### N-{[1-(2-Fluoro-4-trifluoromethylbenzyl)-4-hydroxy-2-oxo-1,2-dihydro-1,8-naphthyridin-3-yl]carbonyl}glycine

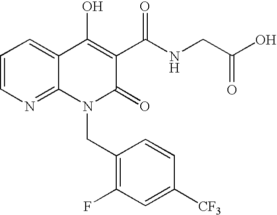

The title compound was prepared using procedures analogous to those described in EXAMPLE 1, substituting 2-fluoro-4-trifluoromethylbenzyl bromide for 4-(trifluoromethyl)benzyl bromide in Step B: MS: m/z 440 (M+H).

### Example 7

### N-({4-Hydroxy-7-methoxy-2-oxo-1-[4-(trifluoromethyl)benzyl]-1,2-dihydro-1,8-naphthyridin-3-yl}carbonyl)glycine

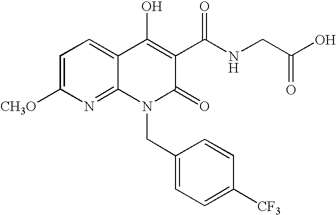

### Step A: 2-Chloro-6-methoxynicotinic acid

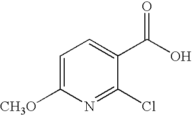

The title compound was prepared according to the procedure of Hirokawa, et. al. (Chem. Phar. Bull., 48, 2000, 1847): ¹H NMR (500 MHz, DMSO-d₆) δ 13.29 (br s, 1H), 8.16 (d, 1H, J=8.4 Hz), 6.90 (d, 1H, J=8.4 Hz), 3.90 (s, 3H); MS: m/z 188 (M+H).

### Step B: Methyl 2-chloro-6-methoxynicotinate

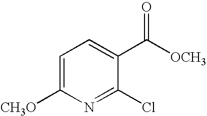

A mixture of 4.8 g (25.6 mmol) of the compound of Step A and thionyl chloride (25 mL, 343 mmol) was refluxed for 5 h. Excess thionyl chloride was removed under vacuum and the mixture was evaporated twice from toluene to afford the intermediate acid chloride. The acid chloride was refluxed with methanol (30 mL) for 1 h and then stirred at ambient temperature for 12 h. The MeOH was removed under vacuum and the residue extracted with chloroform. The solvent layer was washed with water, sat'd NaCl, dried and evaporated. Trituration with hexanes and filtration gave 2.51 g of the title compound as a white solid: ¹H NMR (500 MHz, CDCl₃) δ 8.14 (d, 1H, J=8.5 Hz), 6.72 (d, 1H, J=8.7 Hz), 4.02 (s, 3H), 3.94 (s, 3H).

### Step C: Methyl 6-methoxy-2-{[4-(trifluoromethyl)benzyl]amino}nicotinate

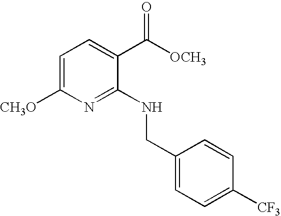

A mixture of 1.0 g (5.0 mmol) of the compound of Step B and 4-(trifluoromethyl)-benzylamine (1.82 g, 10.4 mmol) in DME (25 mL) was refluxed for 48 h. The resulting mixture was filtered and washed with DME. The combined filtrate was evaporated followed by the purification silica column (combiflash, ISCO) and eluted with hexane+EtOAc (0 to 10% gradient) to give 1.49 g of the title compound as an off white solid: ¹H NMR (500 MHz, CDCl₃) δ 8.63 (br s, 1H), 8.04 (d, 1H, J=8.5 Hz), 7.59 (d, 2H, J=7.3 Hz), 7.48 (d, 2H, J=7.2 Hz), 6.02 (d, 1H, J=8.5 Hz), 4.81 (d, 2H, J=5.5 Hz), 3.86 (s, 3H), 3.81 (s, 3H); MS: m/z 341 (M+H).

### Step D: 6-Methoxy-2-{[4-(trifluoromethyl)benzyl]amino}nicotinic acid

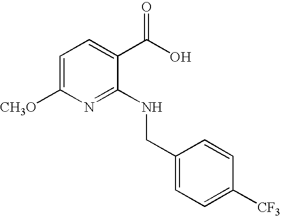

A mixture of 1.0 g (2.9 mmol) of the compound of Step C and sodium hydroxide (0.14 g, 3.53 mmol) in MeOH/water (1:1, 10 mL) was refluxed for 24 h. The resulting mixture was evaporated and the pH of the aqueous mixture was adjusted to pH=3 with 2 N HCl. The solid that precipitated was filtered, washed with water and dried to give 0.97 g of the title compound as a white solid: ¹H NMR (500 MHz, DMSO-d₆) δ 8.78 (t, 1H, J=6 Hz), 7.94 (d, 1H, J=8.5 Hz), 7.65 (d, 2H, J=7.8 Hz), 7.53 (d, 2H, J=7.7 Hz), 5.98 (d, 1H, J=8.5 Hz), 4.74 (d, 2H, J=5.7 Hz), 3.68 (s, 3H); MS: m/z 327 (M+H).

### Step E: 7-Methoxy-1-[4-(trifluoromethyl)benzyl]-2H-pyrido[2,3-d][1,3]oxazine-2,4(1H)-dione

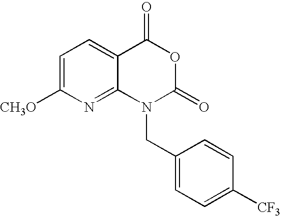

A solution of 0.51 g (1.56 mmol) of the compound of Step D and 0.66 g (6.3 mmol) of sodium carbonate (0.665 g, 6.28 mmol) in water (12 mL) was treated with a solution of phosgene solution (5 mL, 20%) in toluene. The resulting heterogeneous mixture was stirred for 24 h. The solid that was formed was filtered, washed with water and dried to afford 0.21 g of the title compound: ¹H NMR (500 MHz, DMSO-d₆) δ 8.78 (t, 1H, J=6 Hz), 8.22 (d, 1H, J=8.7 Hz), 7.67 (s, 4H), 6.74 (d, 1H, J=8.5 Hz), 5.37 (s, 2H), 3.84 (s, 3H).

### Step F: N-({4-Hydroxy-7-methoxy-2-oxo-1-[4-(trifluoromethyl)benzyl]-1,2-dihydro-1,8-naphthyridin-3-yl}carbonyl)glycine

The title compound was prepared from the compound of Step E using procedures analogous to those described in EXAMPLE 1, Steps C-E: MS: m/z 452 (M+H).

### Example 8

### N-{[1-(1,3-Benzothiazol-2-ylmethyl)-4-hydroxy-2-oxo-1,2-dihydro-1,8-naphthyridin-3-yl]carbonyl}glycine

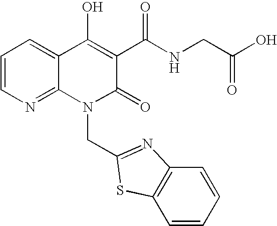

### Step A: 1-(1,3-Benzothiazol-2-ylmethyl)-2H-pyrido[2,3-d][1,3]oxazine-2,4(1H)-dione

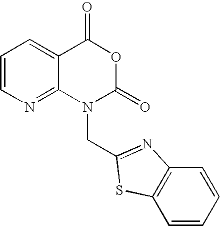

A suspension of 1.0 g (6.1 mmol) of 2H-pyrido[2,3-d][1,3]oxazine-2,4(1H)-dione (from EXAMPLE 1, Step A) and 1.45 g (7.9 mmol) of 2-(chloromethyl)-1,3-benzothiazole (1.45 g, 7.92 mmol) in acetonitrile (40.0 mL) under N₂ was treated with BEMP (2.65 mL, 9.14 mmol) via a syringe and the resulting mixture was stirred at rt for 40 h. The solution was concentration in vacuo and purified on a CombiFlash Companion (40 g column) eluting with 0 to 100% EtOAc/Hexane to afford 0.29 g of the title compound: ¹H NMR (500 MHz, DMSO-d₆) δ 8.77 (dd, 1H, J=1.8, 3.2 Hz), 8.47 (dd, 1H, J=1.8, 5.9 Hz), 8.05 (d, 1H, J=7.8 Hz), 7.94 (d, 1H, J=8 Hz), 7.49˜7.39 (m, 3H), 5.76 (s, 2H).

### Step B: Ethyl 1-(1,3-benzothiazol-2-ylmethyl)-4-hydroxy-2-oxo-1,2-dihydro-1,8-naphthyridine-3-carboxylate

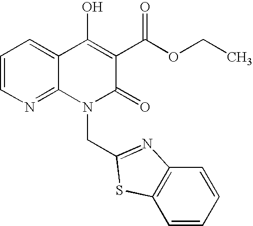

The title compound was prepared using a procedure analogous to that described in EXAMPLE 1, Step C, and the compound from Step A. ¹H NMR (500 MHz, CDCl3) δ 8.73 (d, 1H, J=4.3 Hz), 8.51 (d, 1H, J=7.8 Hz), 8.01 (d, 1H, J=8.0 Hz), 7.79 (d, 1H, J=8.0 Hz), 7.45 (t, 1H, J=7.5 Hz), 7.35 (t, 1H, J=7.7 Hz), 7.29˜7.26 (m, 1H), 6.14 (s, 2H), 4.55 (q, 2H, J=7.1 Hz) 1.51 (t, 3H, J=7.1 Hz); MS: m/z 382.41 (M+H).

### Step C: tert-Butyl N-{[1-(1,3-benzothiazol-2-ylmethyl)-4-hydroxy-2-oxo-1,2-dihydro-1,8-naphthyridin-3-yl]carbonyl}glycinate

The title compound was prepared using a procedure analogous to that described in EXAMPLE 1, Step D, and the compound from Step B. ¹H NMR (500 MHz, DMSO-d₆) δ 10.30 (br s, 1H), 8.78˜8.77 (dd, 1H, J=1.8, 2.8 Hz), 8.54˜8.52 (dd, 1H, J=1.6, 6.1 Hz), 8.00 (d, 1H, J=7.8 Hz), 7.90 (d, 1H, J=8 Hz), 7.48˜7.44 (m, 2H), 7.38 (t, 1H, J=8 Hz), 6.02 (s, 2H), 4.08 (d, 2H, J=5.7 Hz) 1.42 (s, 9H); MS: m/z 467.51 (M+H).

### Step D: N-{[1-(1,3-Benzothiazol-2-ylmethyl)-4-hydroxy-2-oxo-1,2-dihydro-1,8-naphthyridin-3-yl]carbonyl}glycine

The title compound was prepared using a procedure analogous to that described in EXAMPLE 1, Step E, and the compound from Step C. ¹H NMR (500 MHz, DMSO-d₆) δ 12.96˜12.92 (br s, 1H), 10.28 (br d, 1H, J=2.2), 8.79 (d, 1H, J=3.5 Hz), 8.53 (d, 1H, J=7.8 Hz), 8.00 (d, 1H, J=7.8 Hz), 7.90 (d, 1H, J=8 Hz), 7.49˜7.44 (m, 2H), 7.38 (t, 1H, J=7.6 Hz), 6.03 (s, 2H), 4.12 (d, 2H, J=5.5 Hz); MS: m/z 411.40 (M+H).

### Example 9

### N-({4-Hydroxy-6-iodo-2-oxo-1-[4-(trifluoromethyl)benzyl]-1,2-dihydro-1,8-naphthyridin-3-yl}carbonyl)glycine

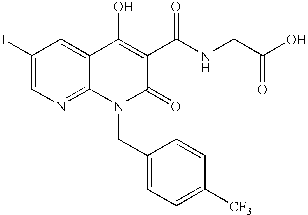

### Step A: Methyl 5-iodo-2-{[4-(trifluoromethyl)benzyl]amino}nicotinate

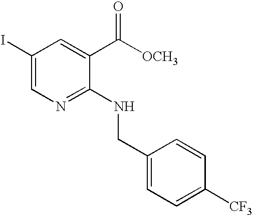

A solution of 1.0 g (3.4 mmol) methyl 5-iodo-2-chloronicotinate and 1.05 mL (7.4 mmol) of 4-(trifluoromethyl)benzylamine in 7.0 mL of ethanol was irradiated in the microwave for a total of 3 h at 140° C. The solvent was evaporated, the residue was triturated with EtOAc and the resulting solid formed was filtered off. The filtrate was concentrated in vacuo to afford a thick oil. The oil was dissolved in minimum amount of EtOAc and filtered through a plug of silica gel washing with 10% EtOAc/hexane. The filtrated was concentrated in vacuo to afford 1.3 g of the title compound: ¹H NMR (500 MHz, CDCl₃) 8.43 (d, 1H, J=1.8 Hz), 8.39 (d, 1H, J=1.9 Hz), 7.59 (d, 2H, J=7.8 Hz), 7.46 (d, 2H, J=7.7 Hz), 4.80 (d, 2H, J=5.7 Hz), 3.91 (s, 3H); MS: m/z 437.17 (M+H).

### Step B: Methyl 2-{(3-ethoxy-3-oxopropanoyl)[4-(trifluoromethyl)benzyl]amino}-5-iodonicotinate

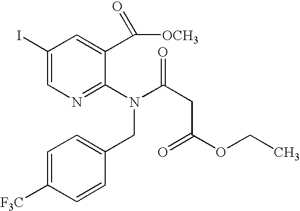

A solution of 1.0 g (2.3 mmol) of the title compound from Step A and pyridine (0.28 mL, 3.44 mmol) in dichloromethane (20 mL) at ambient temperature was treated with ethyl malonyl chloride (0.382 mL, 2.98 mmol) via a syringe and the mixture stirred at 45° C. for 15 h. The mixture was concentrated in vacuo, then purified on the CombiFlash Companion (40 g column) eluting with hexane/EtOAc (gradient, 0% to 45%) to afford 1.05 g of the title compound as an oil: MS: m/z 551.27 (M+H).

### Step C: Ethyl 4-hydroxy-6-iodo-2-oxo-1-[4-(trifluoromethyl)benzyl]-1,2-dihydro-1,8-naphthyridine-3-carboxylate

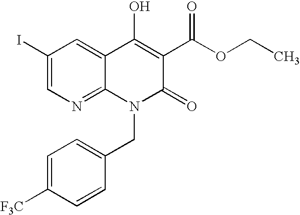

A solution of sodium ethoxide (2.73 mmol) in anhydrous ethanol (5.0 mL) was treated with a solution of 1.0 g (1.8 mmol) of the compound from Step B in 5 mL of ethanol, and the resulting mixture stirred at 60° C. for 30 min. The mixture was cooled to room temperature, quenched with water and concentrated in vacuo. The resulting solid was dissolved in water, diluted with dichloromethane and acidified with 1 N HCl. The layers were separated and the organic layer was dried over magnesium sulfate and concentrated in vacuo. Trituration from ether/hexanes afforded 620 mg of the title compound as a white solid: ¹H NMR (500 MHz, CDCl₃) δ 8.50 (d, 1H, J=2.3 Hz), 8.72 (d, 1H, J=2.3 Hz), 7.56-7.52 (m, 4H), 5.68 (s, 2H), 4.55 (q, 2H, J=7.1 Hz), 1.50 (t, 3H, J=7.0 Hz); MS: m/z 519.23 (M+H).

### Step D: tert-Butyl N-({4-Hydroxy-6-iodo-2-oxo-1-[4-(trifluoromethyl)benzyl]-1,2-dihydro-1,8-naphthyridin-3-yl}carbonyl)glycinate

The title compound was prepared using a procedure analogous to that described in EXAMPLE 1, Step D, and the compound from Step C. ¹H NMR (500 MHz, CDCl₃) δ 10.50 (t, 1H, J=5.3 Hz), 8.86 (d, 1H, J=2.0 Hz), 8.76 (d, 1H, J=1.9 Hz), 7.56-7.52 (m, 4H), 5.73 (s, 2H), 4.15 (d, 2H, J=5.3 Hz), 1.53 (s, 9H); MS: m/z 604.33 (M+H).

### Step E: N-({4-Hydroxy-6-iodo-2-oxo-1-[4-(trifluoromethyl)benzyl]-1,2-dihydro-1,8-naphthyridin-3-yl}carbonyl)glycine

The title compound was prepared using a procedure analogous to that described in EXAMPLE 1, Step E, and the compound from Step D. ¹H NMR (500 MHz, DMSO) δ 12.98-12.92 (br, 1H), 10.26 (s, 1H), 8.95 (d, 1H, J=2.1 Hz), 8.69 (d, 1H, J=1.8 Hz), 7.62 (d, 2H, J=8.0 Hz), 7.62 (d, 2H, J=8.0), 5.64 (s, 2H), 4.12 (d, 2H, J=5.7 Hz); MS: m/z 548.22 (M+H).

### Example 10

### N-({6-Cyano-4-hydroxy-2-oxo-1-[4-(trifluoromethyl)benzyl]-1,2-dihydro-1,8-naphthyridin-3-yl}carbonyl)glycine

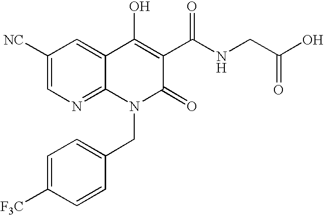

### Step A: tert-Butyl N-({6-cyano-4-hydroxy-2-oxo-1-[4-(trifluoromethyl)benzyl]-1,2-dihydro-1,8-naphthyridin-3-yl}carbonyl)glycinate

A solution of 100 mg (0.17 mmol) of tert-butyl N-({6-iodo-4-hydroxy-2-oxo-1-[4-(trifluoromethyl)benzyl]-1,2-dihydro-1,8-naphthyridin-3-yl}carbonyl)glycinate (from EXAMPLE 9, Step D), 24 mg (0.2 mmol) of zinc cyanide, 30 mg (0.033 mmol) of tris(dibenzylidineacetone)dipalladium(0) and 18.4 mg (0.033 mmol) of 1,1′-bis(diphenylphosphino) in 3 mL of DMF and 0.05 mL water was evacuated of oxygen and the mixture stirred under nitrogen at 110° C. for 2 h. The mixture was cooled, diluted with EtOAc and filtered through celite. The filtrate was concentrated and purified on the CombiFlash Companion (4 g column) eluting with hexane+EtOAc (gradient, 5% to 60% EtOAc) to afford 64 mg of the title compound: ¹H NMR (500 MHz, CDCl₃) δ 10.36 (br s, 1H), 8.92 (d, 1H, J=2.0 Hz), 8.76 (d, 1H, J=2.2 Hz), 7.58˜7.54 (m, 4H), 5.76 (s, 2H), 4.16 (d, 2H, J=5.2 Hz), 1.54 (s, 9H); MS: m/z 503.44 (M+H).

### Step B: N-({6-Cyano-4-hydroxy-2-oxo-1-[4-(trifluoromethyl)benzyl]-1,2-dihydro-1,8-naphthyridin-3-yl}carbonyl)glycine

A solution of 60 mg (0.12 mmol) of the compound from Step A in 5 mL of 4:1 dichloromethane/trifluoroacetic acid was stirred at rt for 20 min. The solution was concentrated and the residue triturated with ether/hexanes to afford 47 mg of the title compound: ¹H NMR (500 MHz, DMSO-d₆) δ 13.02˜12.94 (br s, 1H), 10.18 (br s, 1H), 9.16 (s, 1H), 8.97 (s, 1H), 7.64 (d, 2H, J=8.2 Hz), 7.48 (d, 2H, J=8.0 Hz), 5.91 (s, 2H), 4.13 (d, 2H, J=5.3 Hz); MS: m/z 447.34 (M+H).

The following compounds were prepared in a manner analogous to the procedures described in the previous examples.

### Example 11

### N-({4-Hydroxy-2-oxo-1-[4-(trifluoromethyl)benzyl]-1,2-dihydro-1,8-naphthyridin-3-yl}carbonyl)-L-alanine

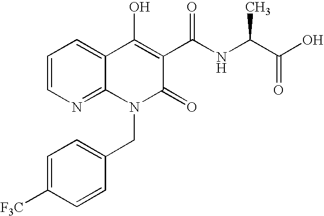

### Example 12

### N-({4-Hydroxy-2-oxo-1-[4-(trifluoromethyl)benzyl]-1,2-dihydro-1,8-naphthyridin-3-yl}carbonyl)-D-alanine

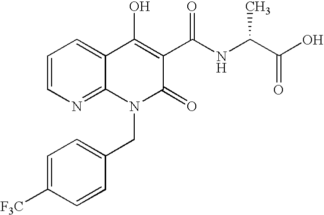

### Example 13

### N-({1-[4-(Aminocarbonyl)benzyl]-4-hydroxy-2-oxo-1,2-dihydro-1,8-naphthyridin-3-yl}carbonyl)glycine

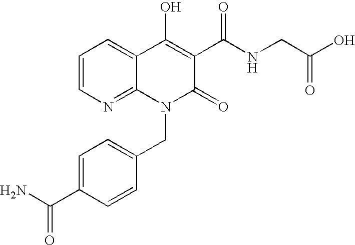

### Example 14

### N-({1-[4-(trifluoromethyl)benzyl]-4-hydroxy-2-oxo-1,2-dihydro-1,8-naphthyridin-3-yl}carbonyl)-L-serine

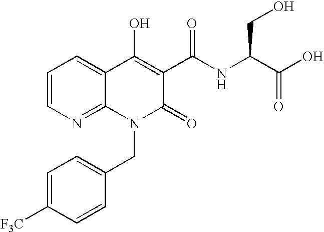

### Example 15

### N-({1-[4-(trifluoromethyl)benzyl]-4-hydroxy-2-oxo-1,2-dihydro-1,8-naphthyridin-3-yl}carbonyl)-L-aspartic acid

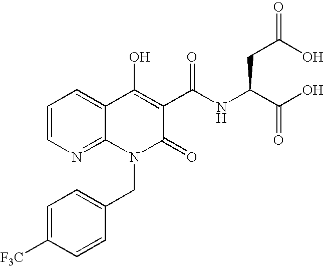

### Example 16

### N-({1-[4-(trifluoromethyl)benzyl]4-hydroxy-2-oxo-1,2-dihydro-1,8-naphthyridin-3-yl}carbonyl)-2-methylalanine

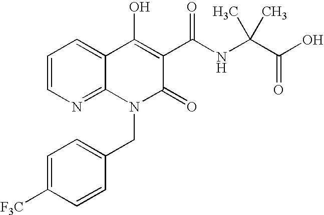

### Example 17

### (2S)-2-[({4-Hydroxy-2-oxo-1-[4-(trifluoromethyl)benzyl]-1,2-dihydro-1,8-naphthyridin-3-yl}carbonyl)amino]butanoic acid

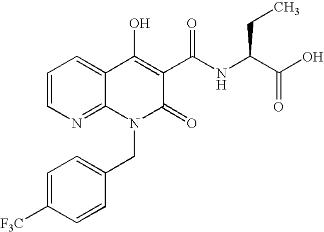

### Example 18

### N-[(4-Hydroxy-2-oxo-1-prop-2-yn-1-yl-1,2-dihydro-1,8-naphthyridin-3-yl)carbonyl]glycine

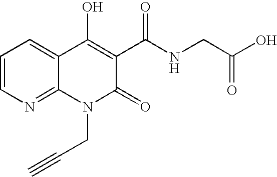

### Example 19

### N-({4-Hydroxy-2-oxo-1-[(3-phenylisoxazol-5-yl)methyl]-1,2-dihydro-1,8-naphthyridin-3-yl}carbonyl)glycine

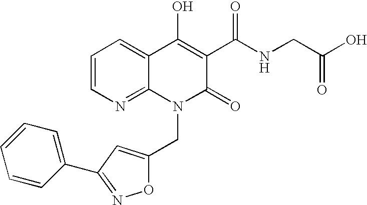

### Example 20

### N-({4-Hydroxy-2-oxo-1-[(5-phenyl-1,2,4-oxadiazol-3-yl)methyl]-1,2-dihydro-1,8-naphthyridin-3-yl}carbonyl)glycine

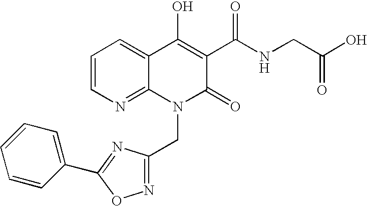

### Example 21

### N-({4-Hydroxy-2-oxo-1-[(3-phenyl-1,2,4-oxadiazol-5-yl)methyl]-1,2-dihydro-1,8-naphthyridin-3-yl}carbonyl)glycine

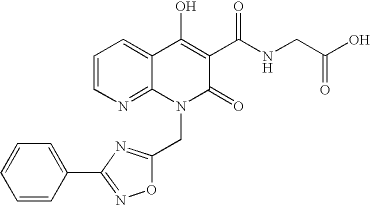

### Example 22

### N-({4-Hydroxy-2-oxo-1-[(4-phenyl-1,3-thiazol-2-yl)methyl]-1,2-dihydro-1,8-naphthyridin-3-yl}carbonyl)glycine

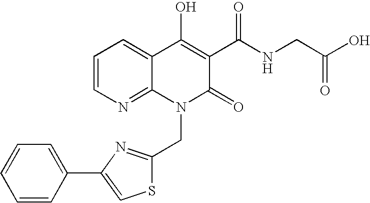

### Example 23

### N-{[1-(2-Ethoxy-2-oxoethyl)-4-hydroxy-2-oxo-1,2-dihydro-1,8-naphthyridin-3-yl]carbonyl}glycine

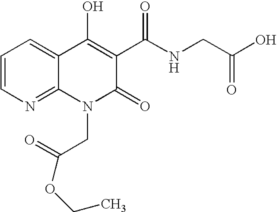

### Example 24

### N-({1-[(5-Chloro-1,3-benzothiazol-2-yl)methyl]-4-hydroxy-2-oxo-1,2-dihydro-1,8-naphthyridin-3-yl}carbonyl)glycine

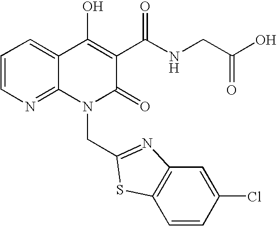

### Example 25

### N-({1-[(4-tert-Butyl-1,3-thiazol-2-yl)methyl]-4-hydroxy-2-oxo-1,2-dihydro-1,8-naphthyridin-3-yl}carbonyl)glycine

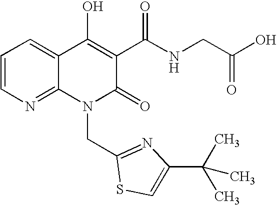

### Example 26

### N-({1-[(4,5-Dimethyl-1,3-thiazol-2-yl)methyl]-4-hydroxy-2-oxo-1,2-dihydro-1,8-naphthyridin-3-yl}carbonyl)glycine

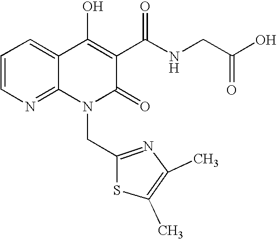

### Example 27

### N-({4-Hydroxy-2-oxo-1-[(5-phenyl-1,3,4-oxadiazol-2-yl)methyl]-1,2-dihydro-1,8-naphthyridin-3-yl}carbonyl)glycine

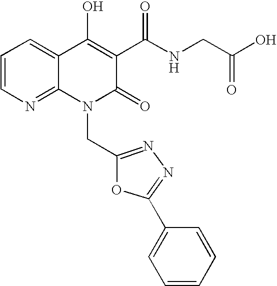

### Example 28

### {[(4-Hydroxy-2-oxo-1-{[6-(trifluoromethyl)pyridin-3-yl]methyl}-1,2-dihydro-1,8 naphthyridin-3-yl) carbonyl}amino}acetic acid

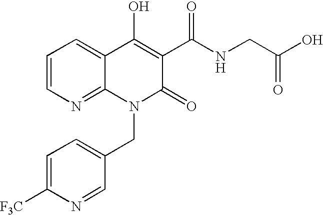

### Step A: 1-{[6-(Trifluoromethyl)pyridin-3-yl]methyl}-2H-pyrido[2,3-d][1,3]oxazine-2,4(1H)-dione

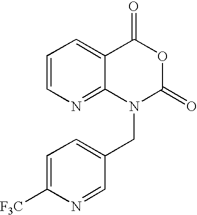

A suspension of 2H-pyrido[2,3-d][1,3]oxazine-2,4(1H)-dione (10.0 g, 60.9 mmol, Step A, Example 1) in DMF (150 mL) at 25° C. was treated with 2.92 g sodium hydride (73.1 mmol, 60% wt. dispersion in mineral oil) and the pale green mixture was stirred at 50° C. for 30 min. The resulting green suspension was cooled to 0° C. and 13.11 g (67.0 mmol) of 5-(chloromethyl)-2-(trifluoromethyl)pyridine was added via a syringe. After stirring at 0° C. for 5 h then at it for 30 min, the mixture was poured into 750 mL of ice water. The solid that precipitated was filtered and dried to afford a pink solid (14.8 g). The solid was added to DCM (300 mL), stirred at rt for 10 min then filtered. The filtrate was concentrated, the residue triturated with ether/hexane, filtered and dried to afford 12.8 g of the title compound: ¹H NMR (500 MHz, CDCl₃) δ 8.98 (s, 1H), 8.76 (dd, 1H, J=4.8, and 1.8 Hz), 8.46 (dd, 1H, J=7.8 and 1.9 Hz), 8.09 (dd, 1H, J=16.1 and 8.1 Hz), 7.67 (d, 1H, J=8.1 Hz), 7.35 (dd, 1H, J=7.8 and 4.8 Hz), 5.57 (s, 2H).

### Step B: Ethyl 4-hydroxy-2-oxo-1-{[6-(trifluoromethyl)pyridin-3-yl]methyl}-1,2-dihydro-1,8-naphthyridine-3-carboxylate

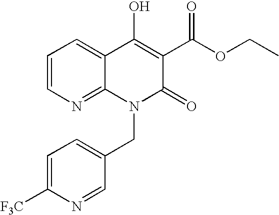

A suspension of 2.22 g (55.4 mmol, 60% wt. dispersion in mineral oil) of sodium hydride in 50 mL of DMF was treated with 9.51 g (59.4 mmol) of diethyl malonate and the resulting mixture was stirred at 60° C. for 15 min. The mixture was then cooled to 0° C. and treated with a solution 12.8 g (39.6 mmol) of 1-{[6-(trifluoromethyl)pyridin-3-yl]methyl}-2H-pyrido[2,3-d][1,3]oxazine-2,4(1H)-dione (from Step A) in 50 mL of DMF via a cannula. The mixture was stirred at 60° C. for 2 h then poured into 500 mL of ice water. The pH of the mixture was adjusted to 6 with 1N HCl and the solid that precipitated was filtered. The solid afforded dissolved in 600 mL of DCM and the resulting solution was washed with water and sat'd NaCl. The organic layer was decolored with charcoal, dried, and filtered through Celite. The filtrate was concentrated and the residue was triturated with ether/hexanes to afford 12.8 g of the title compound: ¹H NMR (500 MHz, CDCl₃) δ 8.93 (s, 1H), 8.70 (dd, 1H, J=4.4, and 1.4 Hz), 8.47 (dd, 1H, J=7.8 and 1.2 Hz), 8.02 (d, 1H, J=7.8 Hz), 7.57 (d, 1H, J=8.0 Hz), 7.27 (m, 1H,), 5.76 (s, 2H), 4.54 (q, 2H, J=14.2 and 7.1 Hz), 1.49 (t, 3H, J=14.2 and 7.1); MS: m/z 394 (M+1).

### Step C: tert-Butyl {[(4-hydroxy-2-oxo-1-{[6-(trifluoromethyl)pyridin-3-yl]methyl}-1,2-dihydro-1,8-naphthyridin-3-yl)carbonyl]amino}acetate

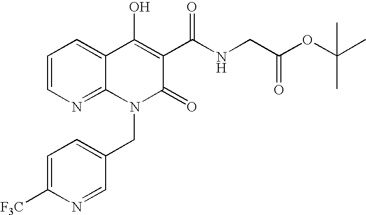

A solution of 12.8 g (32.7 mmol) of ethyl 4-hydroxy-2-oxo-1-{[6-(trifluoromethyl)pyridin-3-yl]methyl}-1,2-dihydro-1,8-naphthyridine-3-carboxylate (from Step B) and 5.31 g (39.2 mmol) of tert-butyl glycine 130 mL of in DME was stirred at 82° C. for 8 h. The residue was triturated with ether/hexane, filtered and dried to afford 14.9 g of the title compound: ¹H NMR (500 MHz, CDCl₃) δ 10.50 (b, 1H), 8.95 (s, 1H), 8.71 (dd, 1H, J=4.6, and 1.9 Hz), 8.49 (dd, 1H, J=8.0 and 1.8 Hz), 8.00 (d, 1H, J=8.0 Hz), 7.59 (d, 1H, J=8.0 Hz), 7.30 (dd, 1H, J=7.8 and 4.6 Hz), 5.80 (s, 2H), 4.15 (d, 2H, J=5.2 Hz), 1.53 (s, 9H). MS: m/z 479 (M+1).

### Step D: {[(4-Hydroxy-2-oxo-1-{[6-(trifluoromethyl)pyridin-3-yl]methyl}-1,2-dihydro-1,8-naphthyridin-3-yl)carbonyl}amino}acetic acid

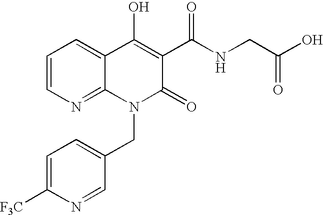

A solution of 14.9 g (31 mmol) of tert-butyl {[(4-hydroxy-2-oxo-1-{[6-(trifluoromethyl)pyridin-3-yl]methyl}-1,2-dihydro-1,8-naphthyridin-3-yl)carbonyl]amino}acetate (from Step C) in 250 mL 4:1 v/v DCM/TFA was stirred at rt for 20 h. The solvent was evaporated. The residue was triturated with ether/hexane (1/4, v/v), filtered, rinsed with water and dried to afford 12.5 g (95%) of the title compound: ¹H NMR (500 MHz, DMSO-d₆) δ 12.98, (b, 1H) 10.28 (t, 1H, J=5.5 Hz), 8.78 (bd, 2H), 8.49 (dd, 1H, J=7.8, and 1.4 Hz), 7.91 (d, 1H, J=8.3 Hz), 7.79 (d, 1H, J=8.0 Hz), 7.45 (dd, 1H, J=8.0 and 4.8 Hz), 5.73 (s, 2H), 4.12 (d, 2H, J=5.5 Hz); MS: m/z 423 (M+1).

### Example 29

### {[(6-Chloro-4-hydroxy-2-oxo-1-{[6-(trifluoromethyl)pyridin-3-yl]methyl}-1,2-dihydro-1,8 naphthyridin-3-yl)carbonyl]amino}acetic acid

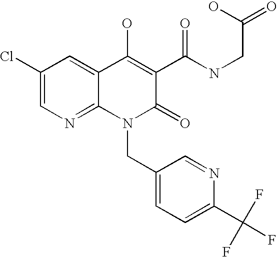

### Step A: Methyl 5-chloro-2-({[6-trifluoromethyl)pyridin-3-yl]methyl}amino)nicotinate

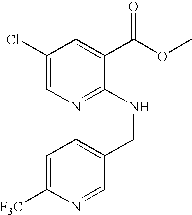

A mixture of 0.30 g (1.46 mmol) of methyl 2,5-dichloronicotinate, 0.31 g (1.75 mmol) of 1-[6-(trifluoromethyl)pyridin-3-yl]methanamine and 0.44 g (4.4 mmol) of TEA in 4 mL of 1,4-dioxane (4.0 mL) was mixture stirred at 100° C. for 30 h. The mixture was cooled and directly purified on the CombiFlash Companion chromatography system eluting with 0-20% EtOAc/hexane gradient to afford 0.29 g of the title compound: ¹H NMR (500 MHz, CDCl₃) δ 8.74, (d, 1H, J=1.2 Hz), 8.44 (t, 1H, J=5.5 Hz), 8.21 (d, 1H, J=2.8 Hz), 8.13 (d, 1H, J=2.5 Hz), 7.85 (dd, 1H, J=8.0 Hz and 1.4 Hz), 7.64 (d, 1H, J=8.0 Hz), 4.83 (d, 2H, J=5.9 Hz), 3.92 (s, 3H); MS: m/z 346 (M+1).

### Step B: Ethyl 6-chloro-4-hydroxy-2-oxo-1-{[6-trifluoromethyl)pyridin-3-yl]methyl}-1,2-dihydro-1,8-naphthyridine-3-carboxylate

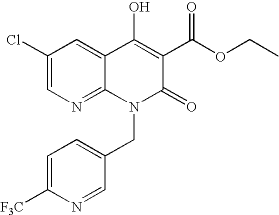

A solution of 0.28 g (0.81 mmol) of methyl 5-chloro-2-({[6-trifluoromethyl)pyridin-3-yl]methyl}amino)nicotinate (from Step A) and 0.13 mL (1.6 mmol) of pyridine in 3 mL of DCM was treated with 0.37 (2.9 mmol) of ethyl 3-chloro-3-oxopropanoate (0.372 mL, 2.916 mmol) and the resulting mixture was stirred at rt for 3 h. The solvent was evaporated and the residue dissolved in 3 mL of absolute EtOH. This solution was added via a syringe this solution was added to 1.2 mmol of freshly prepared sodium ethoxide and the resulting mixture was stirred at 60° C. for 30 min. The reaction was quenched with water (1.0 mL) and concentrated. The resulting solid was redissolved in water/DCM and the pH of the aqueous layer adjusted to pH 5 with 1N HCl. The layers were separated and the organic layer was dried and concentrated. The residue was triturated with ether/hexane (1/2, v/v), filtered and dried to afford 236 mg of the title compound: ¹H NMR (500 MHz, CDCl₃) δ 8.92, (s, 1H), 8.64 (d, 1H, J=2.5 Hz), 8.43 (d, 1H, J=2.5 Hz), 8.00 (d, 1H, J=8.0 Hz), 7.60 (d, 1H, J=8.0 Hz), 5.72 (s, 2H), 4.56 (q, 2H, J=14.4 and 7.1 Hz), 1.51 (t, 3H, J=14.1 and 7.1 Hz); MS: m/z 428 (M+1).

### Step C: tert-Butyl {[(6-Chloro-4-hydroxy-2-oxo-1 {[6-(trifluoromethyl)pyridin-3-yl]methyl}-1,2-dihydro-1,8-naphthyridin-3-yl)carbonyl]amino}acetate

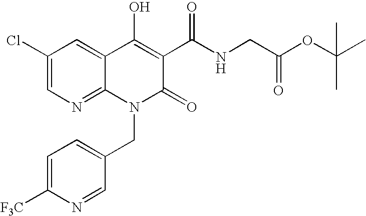

The title compound was prepared from 0.23 g (0.54 mmol) ethyl 6-chloro-4-hydroxy-2-oxo-1-{[6-trifluoromethyl)pyridin-3-yl]methyl}-1,2-dihydro-1,8-naphthyridine-3-carboxylate (from Step B) using a procedure analogous to that described in EXAMPLE 1, Step D: MS: m/z 513 (M+1).

### Step D: {[(6-Chloro-4-hydroxy-2-oxo-1-{[6-(trifluoromethyl)pyridin-3-yl]methyl}-1,2-dihydro-1,8 naphthyridin-3-yl)carbonyl]amino}acetic acid

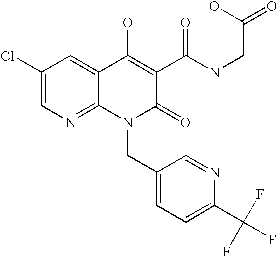

The title compound was prepared from 0.155 g (0.30 mmol) of tert-butyl {[(6-chloro-4-hydroxy-2-oxo-1-{[6-(trifluoromethyl)pyridin-3-yl]methyl}-1,2-dihydro-1,8-naphthyridin-3-yl)carbonyl]amino}acetate (from Step C) using a procedure analogous to that described in EXAMPLE 1, Step E: MS: m/z 457 (M+1).

### Example 30

### (2S)-2-({[1-(1,3-Benzothiazol-2-yl methyl)-4-hydroxy-2-oxo-1,2-dihydro-1,8-naphthyridin-3-yl]carbonyl}amino)propanoic acid

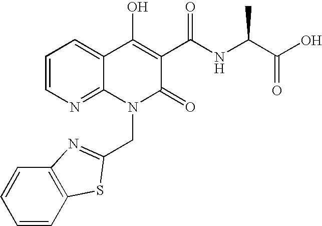

### Example 31

### (2S)-2-[({4-Hydroxy-2-oxo-1-[4-(trifluoromethyl)benzyl]-1,2-dihydro-1,8-naphthyridin-3-yl}carbonyl)amino]succinic acid

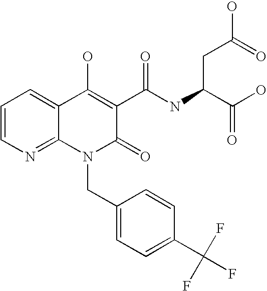

### Example 32

### ({[1-(1,3-Benzothiazol-2-yl methyl)-4-hydroxy-6-iodo-2-oxo-1,2-dihydro-1,8-naphthyridin-3-yl]carbonyl}amino)acetic acid

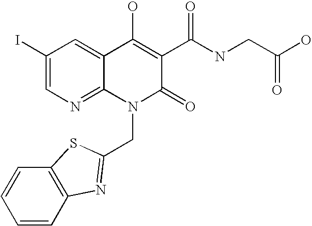

### Example 33

### 2-[({4-Hydroxy-2-oxo-1-[4-(trifluoromethyl)benzyl]-1,2-dihydro-1,8-naphthyridin-3-yl}carbonyl)amino]-2-methylpropanoic acid

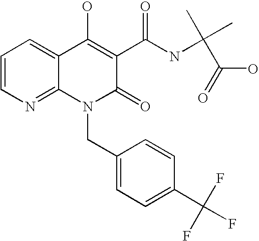

### Example 34

### (2S)-2-[9{4-Hydroxy-2-oxo-1-[4-(trifluoromethyl)benzyl]-1,2-dihydro-1,8-naphthyridin-3-yl}carbonyl]amino]butanoic acid

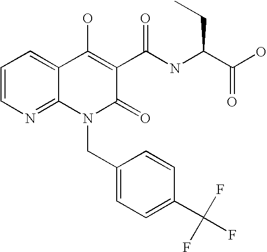

### Example 35

### (2S)-2-[({1-[(5-Chloro-1,3-benzothiazol-2-yl)methyl]-4-hydroxy-2-oxo-1,2-dihydro-1,8-naphthyridin-3-yl}carbonyl)amino]propanoic acid

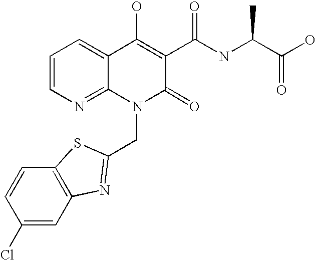

### Example 36

### (2R)-2-[({4-Hydroxy-2-oxo-1-[4-(trifluoromethyl)benzyl]-1,2-dihydro-1,8-naphthyridin-3-yl}carbonyl)amino]succinic acid

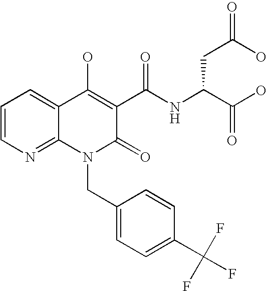

### Example 37

### (2S)-2-({[1-(1,3-Benzothiazol-2-ylmethyl)-4-hydroxy-2-oxo-dihydro-1,8-naphthyridin-3-yl]carbonyl}amino)succinic acid

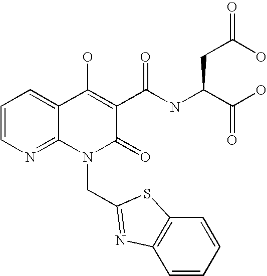

### Example 38

### (2S)-2-{[(4-Hydroxy-2-oxo-1-{[6-(trifluoromethyl)pyridin-3-yl]methyl}-1,2-dihydro-1,8-naphthyridin-3-yl)carbonyl]amino}propanoic acid

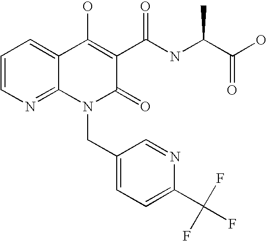

### Example 39

### (2S)-2-[({1-[2-Fluoro-4-(trifluoromethyl)benzyl]-4-hydroxy-2-oxo-1,2-dihydro-1,8-naphthyridin-3-yl}carbonyl)amino]propanoic acid

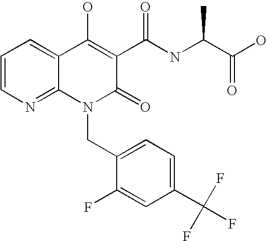

### Example 40

### ({[1-(1,3-Benzothiazol-2-ylmethyl)-6-chloro-4-hydroxy-2-oxo-1,2-dihydro-1,8-naphthyridin-3-yl]carbonyl}amino)acetic acid

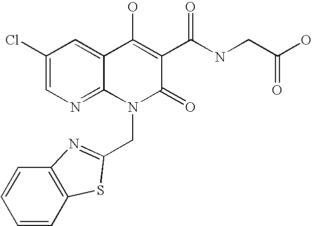

What is claimed is:

1. A compound having the formula I: 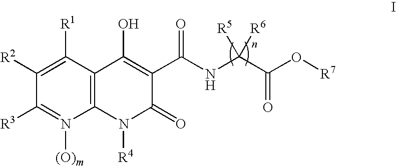
or a pharmaceutically acceptable salt or a solvate thereof, wherein
m is 0 or 1;
n is 1 or 2;
p is 0, 1 or 2;
R¹, R² and R³ are independently selected from the group consisting of:
i) hydrogen,
ii) —C₁-C₁₀ alkyl, optionally substituted with one to five groups independently selected from R^{a}, iii) —C₃-C₁₀ cycloalkyl, optionally substituted with one to five groups independently selected from R^{a},
iv) —C₂-C₁₀ alkenyl, optionally substituted with one to five groups independently selected from R^{a},
v) —C₅-C₁₀ cycloalkenyl, optionally substituted with one to five groups independently selected from R^{a},
vi) —C₂-C₁₀ alkynyl, optionally substituted with one to five groups independently selected from R^{a},
vii) aryl, optionally substituted with one to three groups independently selected from R^{b} and hydroxy,
viii) halogen,
ix) cyano,
x) heteroaryl, optionally substituted with one to three groups independently selected from R^{b},
xi) —O—C₁-C₁₀ alkyl, optionally substituted with one to five groups independently selected from fluorine, hydroxy, oxo, cyano, aryl, substituted aryl, heteroaryl, substituted heteroaryl, —C₁-C₆ alkoxy, aryloxy, substituted aryloxy, heteroaryloxy, substituted heteroaryloxy, —CO₂R⁷, —NR⁸R⁹, —CONR⁸R⁹, —OCO₂R⁷, —OCONR⁸R⁹, —NR¹⁰CO₂R⁷, —NR¹⁰CONR⁸R⁹, and —S(O)ₚR¹⁰;
xii) —O-aryl, optionally substituted with one to three groups independently selected from R^{b} and hydroxy,
xiii) —O-heteroaryl, optionally substituted with one to three groups independently selected from R^{b};
xiv) —SOₚ—C₁-C₁₀ alkyl, optionally substituted with one to five groups independently selected from R^{a};
xvi) —SOₚ-aryl, optionally substituted with one to three groups independently selected from hydroxy and R^{b}; or
R¹ and R², or R² and R³ are joined to form a ring of 5 to 8 atoms optionally substituted with one to three groups independently selected from fluorine, phenyl, substituted phenyl, heteroaryl, substituted heteroaryl, —CONR⁸R⁹, —CO₂R³, and —NR⁸R⁹; where said ring is partially or fully unsaturated having 0, 1 or 2 heteroatoms independently selected from —NR⁷—, —O— and —S(O)ₚ—;
R⁴ is selected from the group consisting of
i) hydrogen;
ii) —C₁-C₁₀ alkyl, optionally substituted with one to five groups independently selected from R^{a};
iii) —(C₀-C₁₀ alkyl)C₃-C₁₀ cycloalkyl, optionally substituted with one to five groups independently selected from R^{a},
iv) —C₂-C₁₀ alkenyl, optionally substituted with one to five groups independently selected from R^{a};
v) —(C₀-C₁₀ alkyl)C₅-C₁₀ cycloalkenyl, optionally substituted with one to five groups independently selected from R^{a};
vi) —C₂-C₁₀ alkynyl optionally substituted with one to five groups independently selected from R^{a};
vii) —(C₀-C₁₀ alkyl)aryl, optionally substituted with one to three groups independently selected from hydroxy and R^{b}; and
ix) —(C₀-C₁₀ alkyl)heteroaryl, optionally substituted with one to three groups independently selected from R^{b};
R⁵ and R⁶ are independently selected from the group consisting of:
i) hydrogen;
ii) C₁-C₄ alkyl, optionally substituted with a hydroxy, —SH, —NH2 or —CO₂H;
iii) trifluoromethyl; and
iv) 2,2,2-trifluoroethyl;
R⁷ is selected from the group consisting of:
i) hydrogen;
ii) —C₁-C₁₀ alkyl;
iii) —(CH₂)₁₋₆—C₃-C₈ cycloalkyl; and
iv) —(CH₂)₁₋₆phenyl;
R⁸, R⁹ and R¹⁰ are independently selected from the group consisting of:
i) hydrogen;
ii) —C₁-C₆ alkyl;
iii) —C₃-C₆ cycloalkyl, wherein alkyl and cycloalkyl are each optionally substituted with one to five groups independently selected from fluorine, hydroxy, oxo, cyano, aryl, substituted aryl, heteroaryl, substituted heteroaryl, —C₁-C₆ alkoxy, substituted —C₁-C₆ alkoxy, aryloxy, substituted aryloxy, heteroaryloxy, substituted heteroaryloxy, —S(O)ₚalkyl and —S(O)ₚaryl;
iv) aryl, optionally substituted with one to three groups independently selected from C₁-C₆ alkyl, halogen, hydroxy, cyano, —CO₂(C₁₋₃alkyl), —CONR¹¹R¹², —OCO₂(C₁₋₃alkyl), —OCONR¹¹R¹², and —S(O)ₚ(C₁₋₃alkyl); and
v) heteroaryl, optionally substituted with one three groups independently selected from C₁-C₆ alkyl, halogen, hydroxy, oxo, cyano, —CO₂(C₁₋₃alkyl), CONR¹¹R¹², —OCO₂(C₁₋₃alkyl), —OCONR¹¹R¹², and —S(O)ₚ(C₁₋₃alkyl); or
R⁸ and R⁹ together with the N atom to which they are attached form a saturated or partially saturated ring of 5 to 8 atoms having 0, 1 or 2 additional heteroatoms selected from —O—, —NR⁷—, and —S(O)ₚ— wherein said ring is optionally substituted with a methyl or hydroxy group;
R¹¹ and R¹² are independently selected from the group consisting of:
i) hydrogen;
ii) C₁-C₄ alkyl, optionally substituted with a hydroxy; or
R¹¹ and R¹² together with the N atom to which they are attached form a saturated or partially saturated ring of 5 to 8 atoms having 0, 1 or 2 additional heteroatoms selected from —O—, —NR⁷—, and —S(O)ₚ—;
R^{a} is selected from the group consisting of fluorine, hydroxy, oxo, cyano, aryl, substituted aryl, heteroaryl, substituted heteroaryl, —C₁-C₆ alkoxy, substituted —C₁-C₆ alkoxy, aryloxy, substituted aryloxy, heteroaryloxy, substituted heteroaryloxy, —CO₂R⁷, —NR⁸R⁹, —CONR⁸R⁹, —OCO₂R⁷, —OCONR⁸R⁹, —NR¹⁰CO₂R⁷, —NR¹⁰CONR⁸R⁹, and —S(O)ₚR¹⁰;
R^{b} is selected from the group consisting of halogen, cyano, aryl, substituted aryl, heteroaryl, substituted heteroaryl, —C₁-C₆ alkyl, substituted —C₁-C₆ alkyl, —C₁-C₆ alkoxy, substituted —C₁-C₆ alkoxy, aryloxy, substituted aryloxy, heteroaryloxy, substituted heteroaryloxy, —CO₂R⁷, —NR⁸R⁹, —CONR⁸R⁹, —OCO₂R⁷, —OCONR⁸R⁹, —NR¹⁰CO₂R⁷, —NR¹⁰CONR⁸R⁹, and —S(O)ₚR¹⁰.

2. A compound of claim 1, or a pharmaceutically acceptable salt or solvate thereof, wherein one of R¹, R² and R³ is hydrogen, and the others are independently selected from i) hydrogen, ii) C₁-C₆ alkyl optionally substituted with one to three groups independently selected from R^{a}, iii) C₃-C₈ cycloalkyl optionally substituted with one to three groups independently selected from C₁-C₄ alkyl, CF₃, and R^{a}, iv) aryl optionally substituted with one or two groups independently selected from hydroxy and R^{b}, v) halogen, vi) cyano, vii) heteroaryl optionally substituted with one or two groups independently selected from R^{b}, viii) —O—C₁-C₆ alkyl optionally substituted with one to three groups independently selected from fluorine, hydroxy, oxo, cyano, aryl, substituted aryl, heteroaryl, substituted heteroaryl, alkoxy, aryloxy, substituted aryloxy, heteroaryloxy, substituted heteroaryloxy, —CO₂R⁷, —NR⁸R⁹, —CONR⁸R⁹, —OCO₂R⁷, —OCONR⁸R⁹, —NR¹⁰CO₂R⁷, —NR¹⁰CONR⁸R⁹, and —S(O)ₚR¹⁰; ix) —O-aryl optionally substituted with one or two groups independently selected from hydroxy and R^{b}, x) —O-heteroaryl optionally substituted with one to two groups independently selected from R^{b}; xi) —SOₚ—C₁-C₆ alkyl optionally substituted with one to three groups independently selected from R^{a}; and xii) —SOₚ-aryl optionally substituted with one to three groups independently selected from hydroxy and R^{b}.

3. A compound of claim 1, or a pharmaceutically acceptable salt or solvate thereof, wherein R¹ is hydrogen, one of R² and R³ is hydrogen and the other is selected from the group consisting of i) hydrogen, ii) halogen, iii) cyano, iv) —C₁-C₃ alkyl optionally substituted with one to three fluorine, and iv) —O—C₁-C₃ alkyl optionally substituted with one to three fluorine.

4. A compound of claim 1, or a pharmaceutically acceptable salt or solvate thereof, wherein R⁴ is —C₁-C₄ alkyl substituted with a group selected from C(O)OH, C(O)O—C₁-C₄ alkyl, aryl, substituted aryl, heteroaryl and substituted heteroaryl.

5. A compound of claim 1, or a pharmaceutically acceptable salt or solvate thereof, where R⁴ is —C₁-C₄ alkyl substituted with phenyl, where phenyl is unsubstituted or substituted with one to three groups independently selected from i) —C₁-C₃ alkyl optionally substituted with one to three fluorine, ii) halogen, iii) cyano, iv) C(O)NH₂, and v) —O—C₁-C₃ alkyl optionally substituted with one to three fluorine.

6. A compound of claim 1, or a pharmaceutically acceptable salt or solvate thereof, where R⁴ is —C₁-C₄ alkyl substituted with heteroaryl, where heteroaryl is unsubstituted or substituted with one to three groups independently selected from i) —C₁-C₄ alkyl optionally substituted with one to three fluorine, ii) halogen, iii) cyano, iv) phenyl, and v) —O—C₁-C₄ alkyl optionally substituted with one to three fluorine.

7. A compound of claim 1 having the formula Ia: 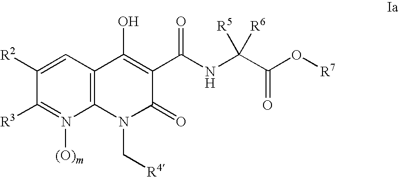
or a pharmaceutically acceptable salt or solvate thereof, wherein m, R², R³, R⁵, R⁶ and R⁷ is as defined in claim 1, and R^{4′} is selected from C(O)OH, C(O)O—C₁-C₄ alkyl, aryl, substituted aryl, heteroaryl and substituted heteroaryl.

8. A compound of claim 7, or a pharmaceutically acceptable salt or solvate thereof, wherein R4′ is selected from i) phenyl optionally substituted with one or two groups independently selected from halogen, cyano, and trifluoromethyl, ii) heteroaryl and substituted heteroaryl selected from benzothiazole, halo-substituted benzothiazole, isoxazole, phenyl substituted isoxazole, 1,2,4-oxadiazole, phenyl substituted 1,2,4-oxadiazole, thiazole, phenyl substituted thiazole, C₁-C₄ alkyl substituted thiazole, di(C₁-C₄)alkyl substituted thiazole, 1,3,4-oxadiazole, and phenyl substituted 1,3,4-oxadiazole.

9. A compound of claim 1 selected from:
N-({4-hydroxy-2-oxo-1-[4-(trifluoromethyl)benzyl]-1,2-dihydro-1,8-naphthyridin-3-yl}carbonyl)glycine;
N-{[1-(4-chlorobenzyl)-4-hydroxy-2-oxo-1,2-dihydro-1,8-naphthyridin-3-yl]carbonyl}glycine;
N-{[1-(4-bromobenzyl)-4-hydroxy-2-oxo-1,2-dihydro-1,8-naphthyridin-3-yl]carbonyl}glycine;
N-{[1-(4-cyanobenzyl)-4-hydroxy-2-oxo-1,2-dihydro-1,8-naphthyridin-3-yl]carbonyl}glycine;
N-{[1-(4-methylbenzyl)-4-hydroxy-2-oxo-1,2-dihydro-1,8-naphthyridin-3-yl]carbonyl}glycine;
N-{[1-(2-fluoro-4-trifluoromethylbenzyl)-4-hydroxy-2-oxo-1,2-dihydro-1,8-naphthyridin-3-yl]carbonyl}glycine;
N-({4-hydroxy-7-methoxy-2-oxo-1-[4-(trifluoromethyl)benzyl]-1,2-dihydro-1,8-naphthyridin-3-yl}carbonyl)glycine;
N-{[1-(1,3-benzothiazol-2-ylmethyl)-4-hydroxy-2-oxo-1,2-dihydro-1,8-naphthyridin-3-yl]carbonyl}glycine;
N-({4-hydroxy-6-iodo-2-oxo-1-[4-(trifluoromethyl)benzyl]-1,2-dihydro-1,8-naphthyridin-3-yl}carbonyl)glycine;
N-({6-cyano-4-hydroxy-2-oxo-1-[4-(trifluoromethyl)benzyl]-1,2-dihydroquinolin-3-yl}carbonyl)glycine;
N-({4-hydroxy-2-oxo-1-[4-(trifluoromethyl)benzyl]-1,2-dihydro-1,8-naphthyridin-3-yl}carbonyl)-L-alanine;
N-({4-hydroxy-2-oxo-1-[4-(trifluoromethyl)benzyl]-1,2-dihydro-1,8-naphthyridin-3-yl}carbonyl)-D-alanine;
N-({1-[4-(aminocarbonyl)benzyl]-4-hydroxy-2-oxo-1,2-dihydro-1,8-naphthyridin-3-yl}carbonyl)glycine;
N-({1-[4-(trifluoromethyl)benzyl]-4-hydroxy-2-oxo-1,2-dihydro-1,8-naphthyridin-3-yl}carbonyl)-L-serine;
N-({1-[4-(trifluoromethyl)benzyl]-4-hydroxy-2-oxo-1,2-dihydro-1,8-naphthyridin-3-yl}carbonyl)-L-aspartic acid;
(2S)-2-[({4-hydroxy-2-oxo-1-[4-(trifluoromethyl)benzyl]-1,2-dihydro-1,8-naphthyridin-3-yl}carbonyl)amino]butanoic acid;
N-[(4-hydroxy-2-oxo-1-prop-2-yn-1-yl-1,2-dihydro-1,8-naphthyridin-3-yl)carbonyl]glycine;
N-({4-hydroxy-2-oxo-1-[(3-phenylisoxazol-5-yl)methyl]-1,2-dihydro-1,8-naphthyridin-3-yl}carbonyl)glycine;
N-({4-hydroxy-2-oxo-1-[(5-phenyl-1,2,4-oxadiazol-3-yl)methyl]-1,2-dihydro-1,8-naphthyridin-3-yl}carbonyl)glycine;
N-({4-hydroxy-2-oxo-1-[(3-phenyl-1,2,4-oxadiazol-5-yl)methyl]-1,2-dihydro-1,8-naphthyridin-3-yl}carbonyl)glycine;
N-({4-hydroxy-2-oxo-1-[(4-phenyl-1,3-thiazol-2-yl)methyl]-1,2-dihydro-1,8-naphthyridin-3-yl}carbonyl)glycine;
N-{[1-(2-ethoxy-2-oxoethyl)-4-hydroxy-2-oxo-1,2-dihydro-1,8-naphthyridin-3-yl]carbonyl}glycine;
N-({1-[(5-chloro-1,3-benzothiazol-2-yl)methyl]-4-hydroxy-2-oxo-1,2-dihydro-1,8-naphthyridin-3-yl}carbonyl)glycine;
N-({1-[(4-tert-butyl-1,3-thiazol-2-yl)methyl]-4-hydroxy-2-oxo-1,2-dihydro-1,8-naphthyridin-3-yl}carbonyl)glycine;
N-({1-[(4,5-dimethyl-1,3-thiazol-2-yl)methyl]-4-hydroxy-2-oxo-1,2-dihydro-1,8-naphthyridin-3-yl}carbonyl)glycine;
N-({4-hydroxy-2-oxo-1-[(5-phenyl-1,3,4-oxadiazol-2-yl)methyl]-1,2-dihydro-1,8-naphthyridin-3-yl}carbonyl)glycine;
{[(4-Hydroxy-2-oxo-1-{[6-(trifluoromethyl)pyridin-3-yl]methyl}-1,2-dihydro-1,8 naphthyridin-3-yl) carbonyl}amino}acetic acid;
{[(6-Chloro-4-hydroxy-2-oxo-1-{[6-(trifluoromethyl)pyridin-3-yl]methyl}-1,2-dihydro-1,8 naphthyridin-3-yl)carbonyl]amino}acetic acid;
(2S)-2-({[1-(1,3-Benzothiazol-2-yl methyl)-4-hydroxy-2-oxo-1,2-dihydro-1,8-naphthyridin-3-yl]carbonyl}amino)propanoic acid;
(2S)-2-[({4-Hydroxy-2-oxo-1-[4-(trifluoromethyl)benzyl]-1,2-dihydro-1,8-naphthyridin-3-yl}carbonyl)amino]succinic acid;
({[1-(1,3-Benzothiazol-2-yl)methyl)-4-hydroxy-6-iodo-2-oxo-1,2-dihydro-1,8-naphthyridin-3-yl]carbonyl}amino)acetic acid;
2-[({4-Hydroxy-2-oxo-1-[4-(trifluoromethyl)benzyl]-1,2-dihydro-1,8-naphthyridin-3-yl}carbonyl)amino]-2-methylpropanoic acid;
(2S)-2-[9{4-Hydroxy-2-oxo-1-[4-(trifluoromethyl)benzyl]-1,2-dihydro-1,8-naphthyridin-3-yl}carbonyl]amino]butanoic acid;
(2S)-2-[({1-[(5-Chloro-1,3-benzothiazol-2-yl)methyl]-4-hydroxy-2-oxo-1,2-dihydro-1,8-naphthyridin-3-yl}carbonyl)amino]propanoic acid;
(2R)-2-[({4-Hydroxy-2-oxo-1-[4-(trifluoromethyl)benzyl]-1,2-dihydro-1,8-naphthyridin-3-yl}carbonyl)amino]succinic acid;
(2S)-2-({[1-(1,3-Benzothiazol-2-ylmethyl)-4-hydroxy-2-oxo-dihydro-1,8-naphthyridin-3-yl]carbonyl}amino)succinic acid;
(2S)-2-{[(4-Hydroxy-2-oxo-1-{[6-(trifluoromethyl)pyridin-3-yl]methyl}-1,2-dihydro-1,8-naphthyridin-3-yl)carbonyl]amino}propanoic acid;
(2S)-2-[({1-[2-Fluoro-4-(trifluoromethyl)benzyl]-4-hydroxy-2-oxo-1,2-dihydro-1,8-naphthyridin-3-yl}carbonyl)amino]propanoic acid; and
({[1-(1,3-Benzothiazol-2-ylmethyl)-6-chloro-4-hydroxy-2-oxo-1,2-dihydro-1,8-naphthyridin-3-yl]carbonyl}amino)acetic acid; or a pharmaceutically acceptable salt or solvate thereof.

10. A method of treating a condition in a mammal, the treatment of which is effected or facilitated by HIE prolyl hydroxylase inhibition, which comprises administering a compound of claim 1, or a pharmaceutically acceptable salt or solvate thereof, in an amount that is effective at inhibiting REF prolyl hydroxylase.

11. A method of claim 10, wherein the condition is anemia.

12. A method of enhancing endogenous production of erythropoietin in a mammal by administering to the mammal an amount of a compound of claim 1, or a pharmaceutically acceptable salt or solvate thereof, that is effective for enhancing endogenous production of erythropoietin.

13. A pharmaceutical composition comprising a pharmaceutically acceptable carrier and a compound claim 1, or a pharmaceutically acceptable salt or solvate thereof.

14. A pharmaceutical composition made by combining the compound of claim 1, or a pharmaceutically acceptable salt or solvate thereof, and a pharmaceutically acceptable carrier.

15. Use of a compound of claim 1, or a pharmaceutically acceptable salt or solvate thereof, in the manufacture of medicaments for the treatment of conditions mediated by HIE prolyl hydroxylase.
